(19) United States
(12) Patent Application Publication
(10) Pub. No.: US 2006/0039975 A1
(43) Pub. Date: Feb. 23, 2006

(54) Paroxetine formulations

(76) Inventors: Zalman Vilkov, Dingmans Ferry, PA (US); Garth Boehm, Westfield, NJ (US)

Correspondence Address: CANTOR COLBURN, LLP, 55 GRIFFIN ROAD SOUTH, BLOOMFIELD, CT, 06002, US

(21) Appl. No.: 11/199,844

(22) Filed: Aug. 9, 2005

Related U.S. Application Data
(60) Provisional application No. 60/603,404, filed on Aug. 20, 2004.

(51) Int. Cl. A61K 9/22; A61K 31/137

(52) U.S. Cl. 424/468; 514/649

(57) ABSTRACT

Paroxetine is a drug used to treat psychiatric problems such as depression. Controlled release dosage forms comprising release-retarding materials other than hydroxypropyl methylcellulose are described. Also described are methods of wet granulating controlled release paroxetine dosage forms.

### CROSS REFERENCE TO RELATED APPLICATIONS

This application claims priority from U.S. Provisional Patent Application No. 60/603,404 filed Aug. 20, 2004, which is hereby incorporated by reference in its entirety.

### BACKGROUND

Paroxetine, (−)-trans-3-[(1,3-benzodioxol-5-yloxy)methyl]-4-(4-fluorophenyl) piperidine; (3S, 4R)-3-[5-(1,3-dioxaindanyl) oxymethyl]-4-(p-fluorophenyl)piperidine, is a 5-hydroxytryptamine (5-HT, serotonin) re-uptake inhibitor

Paroxetine is prescribed for the treatment of psychiatric problems including depression, Parkinson's disease, anxiety disorders, obsessive-compulsive disorders, panic disorder and post-traumatic stress disorder. Other syndromes such as pre-menstrual syndrome (PMS) and male sexual dysfunction may also be treated with paroxetine.

Controlled release paroxetine is marketed as Paxil® CR™ in the U.S. by GlaxoSmithKline. Paxil® CR™ is prescribed as oral dosage tablets containing 12.5 mg, 25 mg or 37.5 mg of the base equivalent of paroxetine hydrochloride. Paxil® CR™ tablets contain a degradable barrier layer and a second layer containing the paroxetine in a hydrophilic matrix. Inactive ingredients include hydroxypropyl methylcellulose, polyvinylpyrrolidone, lactose monohydrate, magnesium stearate, colloidal silicon dioxide, and glyceryl behenate.

The present invention addresses the need for improved paroxetine dosage forms, particularly controlled-release and sustained-release dosage forms.

### SUMMARY OF THE INVENTION

A controlled-release dosage form comprises a pharmaceutically effective amount of paroxetine or a pharmaceutically acceptable salt thereof, and a hydrophilic release-retarding material, wherein the hydrophilic release-retarding material is not hydroxypropyl methylcellulose. The dosage form may exhibit less variability in blood levels than a comparable dose of Paxil® CR™.

A method of forming a controlled-release dosage form comprises wet granulating paroxetine or a pharmaceutically acceptable salt thereof, and a release-retarding material.

These and other advantages of the invention, as well as additional inventive features, will be apparent from the description of the invention provided herein.

### DETAILED DESCRIPTION OF THE INVENTION

The invention relates to improved formulations comprising paroxetine such as, for example, controlled-release formulations, including, but not limited to, wax formulations, press-coated formulations, easily administrable formulations, enteric coated formulations, osmotic pump technology formulations, tamper-resistant formulations, and combination formulations. Solid dosage forms of paroxetine may have a size that is substantially smaller than the size of a same strength dosage form of Paxil® CR™, the marketed form of paroxetine.

One type of formulation is a controlled-release formulation. Controlled-release formulations, such as longer acting formulations that can be administered once daily or even less frequently, are particularly desirable for paroxetine. Controlled-release formulations may provide many inherent therapeutic benefits that are not achieved with corresponding short acting, immediate-release preparations. This is especially true in the treatment of psychiatric problems, where blood levels of paroxetine may be maintained at a therapeutically effective level to provide symptomatic relief. Unless conventional rapid acting drug therapy is carefully administered at frequent intervals to maintain effective steady-state blood levels of the paroxetine, peaks and valleys in the blood level of the paroxetine occur because of paroxetine absorption, systemic excretion of the compound, and through metabolic inactivation, for example, thereby producing special problems in maintaining efficacy. Additionally patient compliance may be improved with dosage formulations that can be administered less frequently.

Controlled-release formulations of the paroxetine may be formulated using OROS (Alza Corp., Mountain View, Calif.) technology. This technology uses osmotic pressure to deliver the paroxetine at a controlled rate. OROS, or osmotic pump, dosage formulations include a semi-permeable membrane surrounding a core that contains at least two components, one component comprising the paroxetine, the other comprising an osmotic push layer, such as an osmotically active polymer. Some time after the dosage form is swallowed, water enters the membrane causing the push layer to swell, releasing the paroxetine at a controlled rate through a hole in the membrane. OROS technology thus may be useful in certain paroxetine formulations.

Sustained-release formulations of paroxetine can be administered once daily or even less frequently. Sustained-release formulations can be based on matrix technology. In this technology, paroxetine is embedded in an excipient that makes a non-disintegrating core called a matrix. Diffusion of paroxetine occurs through the core.

In certain circumstances, delayed-release formulations of paroxetine are desirable. Some active agents, with which the paroxetine may be combined in a combination dosage, cause gastric irritation. Thus controlled-release formulations, such as a delayed-release formulation, with a time-delay before significant plasma levels of the paroxetine are achieved, which avoid an initial burst of the paroxetine, or which are formulated so that release of the paroxetine in the stomach is avoided, may be useful for minimizing these side effects. A delayed-release coated tablet can comprise a core comprising the paroxetine and excipients, optionally free of stabilizer; and a first coating of a water-insoluble, water-permeable film-forming polymer, a plasticizer and a water-soluble polymer, and a second coating of a methacrylic polymer and a plasticizer for the methacrylic polymer. Delayed-release dosage forms, including delayed-release tablets, can exhibit specific dissolution profiles.

Semi-delayed release dosage forms, which are a type of pulsed-release dosage form of paroxetine, are provided. Such dosage forms provide a moderate dosage immediately after administration, and a larger dosage some hours after administration. Such semi-delayed release dosage forms are particularly useful for providing a moderate dosage of paroxetine upon AM administration and a larger dosage at night.

In other circumstances, it may be desirable to precisely control the plasma levels of paroxetine for a number of hours after administration. Pulsed-release formulations, containing some combination of immediate-release, sustained-release, and delayed-release formulations in the same dosage form, can be used in place of multiple immediate and sustained-release dosages in such situations. Other types of pulse-release formulations, which are tailored to provide a particular plasma level profile, are useful in other types of clinical situations.

Enteric coated formulations, which protect the stomach against the irritant effects of paroxetine, are also desirable. Such formulations can be coated with a composition that is non-toxic and includes a pharmaceutically acceptable enteric polymer which is predominantly soluble in the intestinal fluid, but substantially insoluble in the gastric juices.

Another issue is that the current tablet formulations may be inadequate for juvenile and elderly patients who require dosage forms that are easy to swallow. Easily administered formulations, such as chewable tablets, sprinkle forms, liquid formulations, taste-masked formulations, and fast dissolve tablets, are thus desirable.

Patients suffering from psychiatric problems often take multiple medications to effectively control their symptoms or to alleviate side effects. Combinations which contain paroxetine and also contain one or more other active agents typically prescribed for patients suffering from psychiatric problems are for convenient administration and also to improve patient compliance. Thus formulations which incorporate both paroxetine and one or more of other active agents in a single dosage forms are desirable.

Chemical Description and Terminology

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein, the terms wt %, weight percent, percent by weight, etc. are equivalent and interchangeable

The term “active agent” is meant to include solvates (including hydrates) of the free compound or salt, crystalline and non-crystalline forms, as well as various polymorphs. Unless otherwise specified, the term “active agent” is used herein to indicate paroxetine or a pharmaceutically acceptable salt thereof. For example, an active agent can include all optical isomers of the paroxetine and all pharmaceutically acceptable salts thereof either alone or in combination.

“Pharmaceutically acceptable salts” includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

By “water-soluble” active agent is meant an active agent that is at least slightly water-soluble (for example, about 1 to about 10 mg/ml at 25° C.) such as paroxetine. The water solubility of paroxetine is 5.4 mg/ml. Additionally active agents may be moderately water-soluble (for example, less than about 100 mg/ml at 25° C.), or highly water-soluble (for example, greater than about 100 mg/ml at 25° C.).

By “water-insoluble” or “poorly soluble” active agent, it is meant an agent having a water solubility of less than 1 mg/ml, and in some cases even less than 0.1 mg/ml.

By “oral dosage” form is meant to include a unit dosage form prescribed or intended for oral administration. An oral dosage form may or may not comprise a plurality of subunits such as, for example, microcapsules or microtablets, packaged for administration in a single dose.

By “subunit” is meant to include a composition, mixture, particle, etc., that can provide an oral dosage form alone or when combined with other subunits. By “part of the same subunit” is meant to refer to a subunit comprising certain ingredients. For example, a subunit comprising the active agent and an active agent antagonist and/or noxious agent may be placed together with additional subunits in a capsule to provide an oral dosage form.

By “releasable form” is meant to include immediate-release, controlled-release, and sustained-release forms. Certain release forms can be characterized by their dissolution profile. Dissolution profile as used herein, means a plot of the cumulative amount of active ingredient released as a function of time. The dissolution profile can be measured utilizing the Drug Release Test <724>, which incorporates standard test USP 26 (Test <711>). A profile is characterized by the test conditions selected. Thus the dissolution profile can be generated at a preselected apparatus type, shaft speed, temperature, volume, and pH of the dissolution media.

A first dissolution profile can be measured at a pH level approximating that of the stomach. A second dissolution profile can be measured at a pH level approximating that of one point in the intestine or several pH levels approximating multiple points in the intestine.

A highly acidic pH may simulate the stomach and a less acidic to basic pH may simulate the intestine. By the term “highly acidic pH” it is meant a pH of about 1 to about 4. By the term “less acidic to basic pH” is meant a pH of greater than about 4 to about 7.5, preferably about 6 to about 7.5. A pH of about 1.2 can be used to simulate the pH of the stomach. A pH of about 6 to about 7.5, preferably about 6.8, can be used to simulate the pH of the intestine.

Release forms may also be characterized by their pharmacokinetic parameters. “Pharmacokinetic parameters” are parameters which describe the in vivo characteristics of the active agent over time, including for example the in vivo dissolution characteristics and plasma concentration of the active agent. By “Cmax” is meant the measured concentration of the active agent in the plasma at the point of maximum concentration. By “C24” is meant the concentration of the active agent in the plasma at about 24 hours. The term “Tmax” refers to the time at which the concentration of the active agent in the plasma is the highest. “AUC” is the area under the curve of a graph of the concentration of the active agent (typically plasma concentration) vs. time, measured from one time to another.

By “instant-release” is meant a dosage form designed to ensure rapid dissolution of the active agent by modifying the normal crystal form of the active agent to obtain a more rapid dissolution.

By “immediate-release”, it is meant a conventional or non-modified release in which greater then or equal to about 75% of the active agent is released within two hours of administration, preferably within one hour of administration.

By “controlled-release” it is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, i.e., at least over several hours.

“Sustained-release” or “extended-release” include the release of the active agent at such a rate that blood (e.g., plasma) levels are maintained within a therapeutic range, but below toxic levels, for at least about 8 hours, preferably at least about 12 hours after administration at steady-state. The term steady-state means that a plasma level for a given active agent has been achieved and which is maintained with subsequent doses of the drug at a level which is at or above the minimum effective therapeutic level and is below the minimum toxic plasma level for a given active agent. With regard to dissolution profiles, the first and second dissolution profiles (e.g., in the stomach and in the intestines) should each be equal to or greater than the minimum dissolution required to provide substantially equivalent bioavailability to a capsule, tablet or liquid containing the at least one active ingredient in an immediate-release form.

By “delayed-release”, it is meant that there is a time-delay before significant plasma levels of the active agent are achieved. A delayed-release formulation of the active agent can avoid an initial burst of the active agent, or can be formulated so that release of the active agent in the stomach is avoided and absorption is effected in the small intestine.

A “pulsed-release” formulation can contain a combination of immediate-release, sustained-release, and/or delayed-release formulations in the same dosage form. A “semi-delayed-release” formulation is a pulsed-release formulation in which a moderate dosage is provided immediately after administration and a further dosage some hours after administration.

Certain formulations described herein may be “coated”. The coating can be a suitable coating, such as, a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

The term “thermo-responsive” as used herein includes thermoplastic compositions capable of softening, or becoming dispensable in response to heat and hardening again when cooled. The term also includes thermotropic compositions capable of undergoing changes in response to the application of energy in a gradient manner. These compositions are temperature sensitive in their response to the application or withdrawal of energy. Thermo-responsive compositions typically possess the physiochemical property of exhibiting solid, or solid-like properties at temperatures up to about 32° C., and become fluid, semisolid, or viscous when at temperatures above about 32° C., usually in about 32° C. to about 40° C. Thermo-responsive compositions, including thermo-responsive carriers, have the property of melting, dissolving, undergoing dissolution, softening, or liquefying and thereby forming a dispensable composition at the elevated temperatures. The thermo-responsive carrier can be lipophilic, hydrophilic, or hydrophobic. Another property of a thermo-responsive carrier is its ability to maintain the stability of the agent contained therein during storage and during delivery of the agent. A thermo-responsive composition can be easily excreted, metabolized, or assimilated, upon being dispensed into a biological environment.

In some embodiments, the formulations described herein preferably exhibit bioequivalence to the marketed drug product, for example PAXIL® CR™. Bioequivalence is defined as “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study” (21 CFR 320.1). As used herein, bioequivalence of a dosage form is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS-GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001; and “STATISTICAL PROCEDURES FOR BIOEQUIVALENCE STUDIES USING A STANDARD TWO-TREATMENT CROSSOVER DESIGN” DHHS, FDA, CDER, July 1992, all of which are incorporated herein in their entirety.

Particularly relevant sections of the guidelines include:

Pharmacokinetic Analysis of Data: Calculation of area under the plasma concentration-time curve to the last quantifiable concentration (AUC0-t,) and to infinity (AUCO0-∞), Cmax, and Tmax should be performed according to standard techniques.

Statistical Analysis of Pharmacokinetic Data: The log transformed AUC and Cmax data should be analyzed statistically using analysis of variance. These two parameters for the test product should be shown to be within 80-125% of the reference product using the 90% confidence interval. See also Division of Bioequivalence Guidance Statistical Procedures for Bioequivalence Studies Using a Standard Two-Treatment Crossover Design.

Multiple Dose Studies: At a minimum, the following pharmacokinetic parameters for the substance of interest should be measured in a multiple dose bioequivalence study:
a. Area under the plasma/blood concentration—time curve from time zero to time T over a dosing interval at steady state (AUC0-T), wherein T is the dosing interval.
b. Peak drug concentration (Cmax) and the time to peak drug concentration (Tmax), obtained directly from the data without interpolation, after the last dose is administered.
c. Drug concentrations at the end of each dosing interval during steady state (Cmin).
d. Average drug concentration at steady state (Cav), where Cav−AUC0-T/T.
e. Degree of fluctuation (DF) at steady state, where DF=100%×(Cmax−Cmin)/Cav. Evidence of attainment of steady state for the test and reference products should be submitted in the bioequivalence study report.

Statistical Analysis Parametric (normal-theory) general linear model procedures are recommended for the analysis of pharmacokinetic data derived from in vivo bioequivalence studies. An analysis of variance (ANOVA) should be performed on the pharmacokinetic parameters AUC and Cmax using General Linear Models (GLM) procedures of SAS (4) or an equivalent program. Appropriate statistical models pertaining to the design of the bioequivalence study should be employed. For example, for a conventional two-treatment, two-period, two-sequence (2×2) randomized crossover study design, the statistical model often includes factors accounting for the following sources of variation:
1. Sequence (sometimes called Group or Order)
2. Subjects, nested in sequences
3. Period (or Phase)
4. Treatment (sometimes called Drug or Formulation)

The sequence effect should be tested using the [subject (sequence)]mean square from the ANOVA as an error term. All other main effects should be tested against the residual error (error mean square) from the ANOVA. The LSMEANS statement should be used to calculate least squares means for treatments. The ESTIMATE statement in SAS should be used to obtain estimates for the adjusted differences between treatment means and the standard error associated with these differences.

The two one-sided hypotheses at the α=0.05 level of significance should be tested for AUC and Cₘₐₓ by constructing the 90% confidence interval for the ratio between the test and reference averages.

Logarithmic Transformation of Pharmacokinetic Data:

Statistical Assumptions: The assumptions underlying the ANOVA are:
1. Randomization of samples
2. Homogeneity of variances
3. Additivity (linearity) of the statistical model
4. Independency and normality of residuals

In bioequivalence studies, these assumptions can be interpreted as follows:
1. The subjects chosen for the study should be randomly assigned to the sequences of the study.
2. The variances associated with the two treatments, as well as between the sequence groups, should be equal or at least comparable.
3. The main effects of the statistical model, such as 25 subject, sequence, period and treatment effect for a standard 2×2 crossover study, should be additive. There should be no interactions between these effects.
4. The residuals of the model should be independently and normally distributed. In other words, data from bioequivalence studies should have a normal distribution.

If these assumptions are not met, additional steps should be taken prior to the ANOVA including data transformation to improve the fit of the assumptions or use of a nonparametric statistical test in place of ANOVA. However, the normality and constant variance assumptions in the ANOVA model are known to be relatively robust, i.e., small or moderate departure from each (or both) of these assumptions will not have a significant effect on the final result. For all of the disclosed paroxetine dosage forms, bioequivalence to PAXIL CR may be provided according to the FDA guidelines or criteria.

Dosage Forms: Release Properties

The dosage forms comprising paroxetine can be characterized by the release properties of the formulation. Certain dosage form can be targeted-release formulations wherein release occurs in a particular segment of the gastrointestinal tract, for example in the small intestine. Alternatively, the dosage forms can be immediate or modified-release dosage forms in which the rate of release of the active agent within the blood stream is regulated.

Targeted-Release Dosage Forms

Targeted-release refers to release of paroxetine in a particular segment of the gastrointestinal tract. A targeted-release formulation may, for example, have a coat such as an enteric coat, wherein release to a particular portion of the gastrointestinal tract is achieved by the coat. In addition to coatings, other ingredients or techniques may be used to enhance the absorption of the paroxetine, to improve the disintegration profile, and/or to improve the properties of the paroxetine and the like. These include, but are not limited to, the use of additional chemical penetration enhancers, which are referred to herein as noneffervescent penetration enhancers; absorption of the paroxetine onto fine particles to promote absorption by specialized cells within the gastrointestinal tract (such as the M cells of Peyer's patches); ion pairing or complexation; and the use of lipid and/or surfactant paroxetine carriers. The selected enhancement technique is related to the route of active agent absorption, i.e., paracellular or transcellular.

A bioadhesive polymer may be included in the oral dosage form to increase the contact time between the dosage form and the mucosa of the most efficiently absorbing section of the gastrointestinal tract. Nonlimiting examples of known bioadhesives include carbopol (various grades), sodium carboxy methylcellulose, methylcellulose, polycarbophil (NOVEON AA-1), hydroxypropyl methylcellulose, hydroxypropyl cellulose, sodium alginate, sodium hyaluronate, and combinations comprising one or more of the foregoing bioadhesives.

Disintegration agents may also be employed to aid in dispersion of the paroxetine in the gastrointestinal tract. Disintegration agents may be pharmaceutically acceptable effervescent agents. In addition to the effervescence-producing disintegration agents, a dosage form may include suitable noneffervescent disintegration agents. Nonlimiting examples of disintegration agents include microcrystalline cellulose, croscarmelose sodium, crospovidone, sodium starch glycolate, starches and modified starches, and combinations comprising one or more of the foregoing disintegration agents.

Apart from any effervescent material within the tablet, additional effervescent components or, alternatively, only sodium bicarbonate (or other alkaline substance) may be present in the coating around the dosage form. The purpose of the latter effervescent/alkaline material is to react within the stomach contents and promote faster stomach emptying.

Enteric Coated Formulations

An enteric coating is a coating that prevents release of the active agent until the dosage form reaches the small intestine. Enteric coated dosage forms comprise paroxetine coated with an enteric polymer. The enteric polymer may be predominantly soluble in the intestinal fluid, but substantially insoluble in the gastric juices. Examples include polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, SANDARAC, copal collophorium, and combinations comprising one or more of the foregoing polymers. Yet other examples of enteric polymers include synthetic resin bearing carboxyl groups. The methacrylic acid: acrylic acid ethyl ester 1:1 copolymer solid substance of the acrylic dispersion sold under the trade designation “EUDRAGIT L-100-55” may be suitable.

In one embodiment, the enteric coating may comprise about 40 wt % to about 95 wt % enteric polymer (e.g., EUDRAGIT L30D-55) and about 5 wt % to about 60 wt % plasticizer (e.g., triethyl citrate, polyethylene glycol) based on the total weight of the enteric coating. The relative proportions of ingredients, notably the ratio of methacrylic polymer to plasticizer can be varied according to a methods known to those of skill in the art of pharmaceutical formulation.

Immediate-Release Dosage Forms

An immediate-release dosage form is one in which the release properties of the paroxetine from the dosage form are essentially unmodified. An immediate-release dosage form preferably results in delivery of greater then or equal to about 75 wt % the paroxetine within about 2 hours of administration, preferably within 1 hour of administration. An immediate-release dosage form may contain optional excipients so long as the excipients do not significantly extend the release time of the paroxetine.

Controlled Release Dosage Forms

One controlled-release dosage form comprises paroxetine and a hydrophilic release-retarding agent. Suitable controlled release dosage forms comprise 12.5 g, 25 g or 35 g of paroxetine. The hydrophilic release-retarding agent preferably does not comprise hydroxypropyl methylcellulose. Hydrophilic polymers useful for forming a hydrophilic matrix include ethylcellulose, hydroxypropyl cellulose (HPC), hydroxyethyl cellulose (HEC), polyethylene oxide (PEO), polyvinyl alcohol (PVA), xanthan gum, carbomer, carrageenan, and combinations comprising one or more of the foregoing hydrophilic polymers.

In one embodiment, the controlled-release dosage form comprising paroxetine is formed by wet granulation. The term wet granulation refers to granulation in the presence of an aqueous processing solvent, as opposed to granulation in the absence of a processing solvent or a non-aqueous processing solvent. The processing solvent of the wet granulation may be water or a mixture of water and another water miscible solvent. Examples of such water miscible solvents include C1 to C3 alcohols and lower ketones. Suitable solvent mixtures include, for example, water and acetone; water and methanol; water and ethanol; and water, methanol/ethanol and acetone.

In one embodiment, paroxetine or a salt such as the hydrochloride hemihydrate, a release-retarding agent, and other ingredients are be mixed together in the wet granulation process. A high sheer mixer may be used. Water is added to the mixer to start the wet granulation process. After the granulation process, the granulate is milled to obtain a granulate of a desired size. Additional amounts of filler and disintegrant can be added while using a mixer, such as a tumbler mixer. The excipients added after granulation can be of the same or different quality/grade of the same excipient used during the granulation process. After wet granulation, the granulate is optionally dried. A lubricant, such as magnesium stearate, may be added to obtain a final blend. The resulting final blend may then be compressed into a tablet.

Sustained-Release Dosage Forms

A sustained-release form is a dosage form suitable for providing controlled-release of the paroxetine over a sustained period of time (e.g., 8 hours, 12 hours, 24 hours). Sustained-release dosage forms of the paroxetine may release the paroxetine at a rate independent of pH, for example, about pH 1.2 to about 7.5. Alternatively, sustained-release dosage forms may release the paroxetine at a rate dependent upon pH, for example, a lower rate of release at pH 1.2 and a higher rate of release at pH 7.5. The sustained-release form may avoid dose dumping upon oral administration. The sustained-release oral dosage form can be formulated to provide for an increased duration of paroxetine action allowing once-daily dosing.

A sustained-release dosage form comprises a release-retarding material. The release-retarding material can be, for example, in the form of a matrix or a coating. The paroxetine in sustained-release form may be, for example, a particle of the paroxetine that is combined with a release-retarding material. The release-retarding material is a material that permits release of the active agent at a sustained rate in an aqueous medium. The release-retarding material can be selectively chosen so as to achieve, in combination with the other stated properties, a desired in vitro release rate.

Release-retarding materials can be hydrophilic and/or hydrophobic polymers. Release-retarding materials include, for example acrylic polymers, alkylcelluloses, shellac, zein, hydrogenated vegetable oil, hydrogenated castor oil, and combinations comprising one or more of the foregoing materials. The oral dosage form can contain about 1 wt % to about 80 wt % of the release-retarding material based on the total weight of the oral dosage form. Suitable acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and combinations comprising one or more of the foregoing polymers. The acrylic polymer may comprise a methacrylate copolymers described in NF XXIV as fully polymerized copolymers of acrylic and methacrylic acid esters with a low content of quaternary ammonium groups.

Suitable alkylcelluloses include, for example, ethylcellulose and hydroxyethyl cellulose. Those skilled in the art will appreciate that other cellulosic polymers, including other alkyl cellulosic polymers, can be substituted for part or all of the ethylcellulose.

Other suitable hydrophobic materials are water-insoluble with more or less pronounced hydrophobic trends. The hydrophobic material may have a melting point of about 30° C. to about 200° C., or about 45° C. to about 90° C. The hydrophobic material can include neutral or synthetic waxes, fatty alcohols (such as lauryl, myristyl, stearyl, cetyl or preferably cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations comprising one or more of the foregoing materials. Suitable waxes include beeswax, glycowax, castor wax, carnauba wax and wax-like substances, e.g., materials normally solid at room temperature and having a melting point of about 30° C. to about 100° C., and combinations comprising one or more of the foregoing waxes.

In other embodiments, the release-retarding material may comprise digestible, long chain (e.g., C8-C50, preferably C12-C40), substituted or unsubstituted hydrocarbons, such as fatty acids, fatty alcohols, glyceryl esters of fatty acids, mineral and vegetable oils, waxes, and combinations comprising one or more of the foregoing materials. Hydrocarbons having a melting point of about 25° C. and about 90° C. may be used. Of these long chain hydrocarbon materials, fatty (aliphatic) alcohols may be employed. The oral dosage form can contain up to about 60 wt % of a digestible, long chain hydrocarbon, based on the total weight of the oral dosage form.

Further, the sustained-release matrix can contain up to about 60 wt % of a polyalkylene glycol.

Alternatively, the release-retarding material may comprise polylactic acid, polyglycolic acid, or a co-polymer of lactic and glycolic acid.

Release-modifying agents, which affect the release properties of the release-retarding material, may optionally be used. The release-modifying agent may, for example, function as a pore-former. The pore former can be organic or inorganic, and includes materials that can be dissolved, extracted or leached from the coating into the environment of use. The pore-former can comprise one or more hydrophilic polymers, such as hydroxypropyl methylcellulose, hydroxypropyl cellulose, polycarbonates comprised of linear polyesters of carbonic acid in which carbonate groups reoccur in the polymer chain, and combinations comprising one or more of the foregoing release-modifying agents. Alternatively, the pore-former may be a small molecule such as lactose, or metal stearates, and combinations comprising one or more of the foregoing release-modifying agents.

The release-retarding material can also optionally include other additives such as an erosion-promoting agent (e.g., starch and gums); and/or a semi-permeable polymer. In addition to the above ingredients, a sustained-release dosage form may also contain suitable quantities of other materials, e.g., diluents, lubricants, binders, granulating aids, colorants, flavorants and glidants that are conventional in the pharmaceutical art. The release-retarding material can also include an exit means comprising at least one passageway, orifice, or the like. The passageway can have any shape, such as round, triangular, square, elliptical, irregular, etc.

The sustained-release dosage form comprising paroxetine and a release-retarding material may be prepared by a suitable technique for preparing active agents as described in detail below. The paroxetine and release-retarding material may, for example, be prepared by wet granulation techniques, melt extrusion techniques, etc. To obtain a sustained-release dosage form, it may be advantageous to incorporate an additional hydrophobic material.

The paroxetine in sustained-release form can include a plurality of substrates comprising the paroxetine, which substrates are coated with a sustained-release coating comprising a release-retarding material. The sustained-release preparations may thus be made in conjunction with a multiparticulate system, such as beads, ion-exchange resin beads, spheroids, microspheres, seeds, pellets, granules, and other multiparticulate systems in order to obtain a desired sustained-release of the paroxetine. The multiparticulate system can be presented in a capsule or other suitable unit dosage form.

In certain cases, more than one multiparticulate system can be used, each exhibiting different characteristics, such as pH dependence of release, time for release in various media (e.g., acid, base, simulated intestinal fluid), release in vivo, size, and composition.

In some cases, a spheronizing agent, together with the paroxetine can be spheronized to form spheroids. Microcrystalline cellulose and hydrous lactose impalpable are examples of such agents. Additionally (or alternatively), the spheroids can contain a water insoluble polymer, such as an acrylic polymer, an acrylic copolymer, such as a methacrylic acid-ethyl acrylate copolymer, or ethyl cellulose. In this formulation, the sustained-release coating will generally include a water insoluble material such as a wax, either alone or in admixture with a fatty alcohol, or shellac or zein.

Spheroids or beads, coated with paroxetine can be prepared, for example, by dissolving or dispersing the paroxetine in a solvent and then spraying the solution onto a substrate, for example, sugar spheres NF, 18/20 mesh, using a Wurster insert. Optionally, additional ingredients may be also added prior to coating the beads in order to assist the paroxetine binding to the substrates, and/or to color the resulting beads, etc. The resulting substrate-paroxetine may optionally be overcoated with a barrier material, to separate the therapeutically active agent from the next coat of material, e.g., release-retarding material. The barrier material may be a material comprising hydroxypropyl methylcellulose. However, film-formers known in the art may be used. Preferably, the barrier material may not affect the dissolution rate of the final product.

To obtain a sustained-release of the active agent in a manner sufficient to provide a therapeutic effect for the sustained duration, the substrate comprising the paroxetine can be coated with an amount of release-retarding material sufficient to obtain a weight gain level of about 2 wt % to about 30 wt %, although the coat can be greater or lesser depending upon the physical properties of the active agent utilized and the desired release rate, among other things. Moreover, there can be more than one release-retarding material used in the coat, as well as various other pharmaceutical excipients.

The release-retarding material may thus be in the form of a film coating comprising a dispersion of a hydrophobic polymer. Solvents used for application of the release-retarding coating include pharmaceutically acceptable solvents, such as water, methanol, ethanol, methylene chloride, and combinations comprising one or more of the foregoing solvents.

In addition, the sustained-release profile of paroxetine release in the formulations (either in vivo or in vitro) can be altered, for example, by using more than one release-retarding material, varying the thickness of the release-retarding material, changing the particular release-retarding material used, altering the relative amounts of release-retarding material, altering the manner in which the plasticizer is added (e.g., when the sustained-release coating is derived from an aqueous dispersion of hydrophobic polymer), by varying the amount of plasticizer relative to retardant material, by the inclusion of additional ingredients or excipients, by altering the method of manufacture, etc.

In addition to or instead of being present in a matrix, the release-retarding agent can be in the form of a coating. Optionally, the dosage forms can be coated, or a gelatin capsule can be further coated, with a sustained-release coating such as the sustained-release coatings described herein. Such coatings are particularly useful when the subunit comprises the paroxetine in releasable form, but not in sustained-release form. The coatings preferably include a sufficient amount of a hydrophobic material to obtain a weight gain level of about 2 wt % to about 30 wt %, although the overcoat can be greater upon the physical properties of the particular the active agent and the desired release rate, among other things.

The sustained-release formulations preferably may release the paroxetine, e.g., when ingested and exposed to gastric fluids, and then to intestinal fluids. The sustained-release profile of the formulations can be altered, for example, by varying the amount of retardant, e.g., hydrophobic material, by varying the amount of plasticizer relative to hydrophobic material, by the inclusion of additional ingredients or excipients, by altering the method of manufacture, etc.

Delayed-Release Dosage Forms

Delayed-release tablets can comprise a core, a first coating and optionally a second coating. The core may include the paroxetine, and excipients, notably a lubricant, and a binder and/or a filler, and optionally a glidant as well as other excipients.

Examples of suitable lubricants include stearic acid, magnesium stearate, glyceryl behenate, talc, mineral oil (in PEG), and combinations comprising one or more of the foregoing lubricants. Examples of suitable binders include water-soluble polymers, such as modified starch, gelatin, polyvinylpyrrolidone, polyvinyl alcohol, and combinations comprising one or more of the foregoing binders. Examples of suitable fillers include lactose, microcrystalline cellulose, and combinations comprising one or more the foregoing fillers. An example of a glidant is silicon dioxide (AEROSIL, Degussa).

The core may comprise, by dry weight, about 0.1 wt % to about 98 wt % paroxetine, or a pharmaceutically acceptable salt thereof, about 0.5 wt % to about 10 wt % lubricant, and about 2 wt % to about 98 wt % binder or filler.

The first coating may be, for example, a semi-permeable coating to achieve delayed release of the paroxetine. The first coating may comprise a water-insoluble, film-forming polymer, together with a plasticizer and a water-soluble polymer. The water-insoluble, film-forming polymer can be a cellulose ether, such as ethylcellulose, a cellulose ester, such as cellulose acetate, polyvinylalcohol, etc. A suitable film-forming polymer is ethylcellulose (available from Dow Chemical under the trade name ETHOCEL). Other excipients can optionally also be present in the first coating, as for example acrylic acid derivatives (such and EUDRAGIT, Rohm Pharma), pigments, etc.

The first coating comprises about 20 wt % to about 85 wt % water-insoluble, polymer (e.g. ethylcellulose), about 10 wt % to about 75 wt % water-soluble polymer (e.g. polyvinylpyrrolidone), and about 5 wt % to about 30 wt % plasticizer based on the total weight of the first coating. The relative proportions of ingredients, notably the ratio of water-insoluble, film-forming polymer to water-soluble polymer, can be varied depending on the release profile to be obtained (where a more delayed release is generally obtained with a higher amount of water-insoluble, film-forming polymer).

The weight ratio of first coating to tablet core can be about 1:30 to about 3:10, specifically about 1:10.

The optional second coating may be designed to protect the coated tablet core from coming into contact with gastric juice, thereby preventing a food effect. The second coating may comprises an enteric polymer of the methacrylic type and optionally a plasticizer. The second coating can contain about 40 wt % to about 95 wt % enteric polymer (e.g., EUDRAGIT L30D-55) and about 5 wt % to about 60 wt % plasticizer (e.g., triethyl citrate, polyethylene glycol) based on the total weight of the second coating. The relative proportions of ingredients, notably the ratio methacrylic polymer to plasticizer can be varied according to a methods known to those of skill in the art of pharmaceutical formulation.

A process for preparing a delayed-release dosage form of the paroxetine comprises manufacturing a core by, for example, wet or dry granulation techniques. Alternatively, the paroxetine and lubricant may be mixed in a granulator and heated to the melting point of the lubricant to form granules. This mixture can then be mixed with a suitable filler and compressed into tablets. Alternatively, the paroxetine and a lubricant (e.g., mineral oil in PEG) may be mixed in a granulator, e.g., a fluidized bed granulator, and then into tablets. Tablets may be formed by standard techniques, e.g. on a (rotary) press (for example KILIAN) fitted with suitable punches. The resulting tablets are hereinafter referred as tablet cores.

The coating process can be as follows. Ethylcellulose and polyethylene glycol (e.g., PEG 1450) are dissolved in a solvent such as ethanol; polyvinylpyrrolidone is then added. The resulting solution is sprayed onto the tablet cores, using a coating pan or a fluidized bed apparatus to form a first coating.

The process for applying the second coating can be as follows. Triethyl citrate and polyethylene glycol (e.g. PEG 1450) may be dissolved in a solvent such as water; methacrylic polymer dispersion is then added. If present, silicon dioxide can be added as a suspension. The resulting solution may be sprayed onto the coated tablet cores, using a coating pan or a fluidized bed apparatus.

The weight ratio of the second coating to coated tablet core is about 1:30 to about 3:10, specifically about 1:10.

An exemplary delayed release dosage form comprises a core containing the paroxetine, polyvinylalcohol and glyceryl behenate; a first coating of ethylcellulose, polyvinylpyrrolidone and polyethylene glycol, and a second coating of methacrylic acid co-polymer type C, triethyl citrate, polyethylene glycol and optionally containing silicon dioxide.

Pulsed-Release Dosage Forms

An exemplary pulsed-release dosage form may provide at least a part of the dose with a pulsed delayed-release of the drug and another part of the formulation with rapid or immediate-release. The immediate and pulsed delayed-release of the paroxetine can be achieved according to different principles, such as by single dose layered pellets or tablets, by multiple dose layered pellets or tablets, or by two or more different fractions of single or multiple dose layered pellets or tablets, optionally in combination with pellets or tablets having instant release. Multiple dose layered pellets may be filled into a capsule or together with tablet excipients compressed into a multiple unit tablet. Alternatively, a multiple dose layered tablet may be prepared.

Single dose layered pellets or tablets giving one single delayed-release pulse of the paroxetine may be prepared. The single dose layered pellets or tablets may comprise a core material, optionally layered on a seed/sphere, the core material comprising the paroxetine together with a water swellable substance; a surrounding lag time controlling layer, and an outer coating layer positioned to cover the lag time controlling layer. Alternatively, the layered pellets or tablets may comprise a core material comprising the paroxetine; a surrounding layer comprising a water swellable substance; a surrounding lag time controlling layer; and an outer coating layer positioned to cover the lag time controlling layer.

Multiple dose layered pellets or tablets giving two or more delayed-release pulses of the paroxetine may be prepared comprising a core material, optionally layered on a seed/sphere comprising the paroxetine and a water swellable substance, a surrounding lag time controlling layer, a layer comprising the paroxetine optionally together with a water swellable substance; optionally a separating layer which is water-soluble or rapidly disintegrating; and an outer coating layer. Alternatively, multiple dose layered pellets or tablets may comprise a core material, optionally layered on a seed/sphere, comprising the paroxetine; a surrounding layer comprising a water swellable substance; a surrounding lag time controlling layer; a layer comprising the paroxetine; optionally a separating layer; and an outer coating layer.

The core material comprising the paroxetine can be prepared either by coating or layering the paroxetine onto a seed, such as for instance sugar spheres, or by extrusion/spheronization of a mixture comprising the paroxetine and pharmaceutically acceptable excipients. It is also possible to prepare the core material by using tablet technology, i.e., compression of paroxetine granules and optionally pharmaceutically acceptable excipients into a tablet core. For pellets of the two types, i.e., single or multiple dose pellets, which have the paroxetine deposited onto a seed/sphere by layering, it is also possible to have an optional layer comprising a water swellable substance beneath the paroxetine containing layer in the core material. The seeds/spheres can be water insoluble and comprise different oxides, celluloses, organic polymers and other materials, alone or in mixtures, or be water soluble and comprise different inorganic salts, sugars and other materials, alone or in mixtures. Further, the seeds/spheres may comprise paroxetine in the form of crystals, agglomerates, compacts etc. The size of the seeds may be about 0.1 mm to about 2 mm. Before the seeds are layered, the paroxetine may be mixed with further components to obtain preferred handling and processing properties and a suitable concentration of the active substance in the final mixture.

Optionally an osmotic agent is placed in the core material. Such an osmotic agent is water soluble and may provide an osmotic pressure in the tablet. Examples of osmotic agents are magnesium sulfate, sodium chloride, lithium chloride, potassium chloride, potassium sulfate, sodium carbonate, lithium sulfate, calcium bicarbonate, sodium sulfate, calcium lactate, urea, magnesium succinate, sucrose, and combinations comprising one or more of the foregoing osmotic agents.

Water swellable substances suitable for the dosage forms are compounds which are able to expand when they are exposed to an aqueous solution, such as gastro-intestinal fluid. One or more water swellable substances may be present in the core material together with the paroxetine and optionally pharmaceutically acceptable excipient(s). Alternatively, one or more water swellable substances are included in a swelling layer applied onto the core material. As a further alternative, swellable substances(s) may also be present in an optional swelling layer situated beneath the paroxetine containing layer, if a layered seed or sphere is used as the core material.

The amount of water swellable substance(s) in the swelling layer or in the core to material ratio is chosen in such a way that the core material or the swelling layer in contact with an aqueous solution, such as gastro-intestinal fluid, will expand to such a degree that the surrounding lag-time controlling membrane ruptures. A water swellable substance may also be included in the drug comprising layer of the multiple layered pellets or tablets to increase dissolution rate of the drug fraction.

Suitable substances which can be used as water swellable substances include, for example, low-substituted hydroxypropyl cellulose, e.g., L-HPC; cross-linked polyvinyl pyrrolidone (PVP-XL), e.g., Kollidon® CL and Polyplasdone® XL; cross-linked sodium carboxymethylcellulose, e.g., Ac-di-sole, Primellose®; sodium starch glycolate, e.g., Primojel®; sodium carboxymethylcellulose, e.g., Nymcel ZSB10®; sodium carboxymethyl starch, e.g., Explotab®; ion-exchange resins, e.g., Dowex® or Amberlite®; microcrystalline cellulose, e.g., Avicel®; starches and pregelatinized starch, e.g., Starch 1500®, Sepistab ST200®; formalin-casein, e.g., Plas-Vita®, and combinations comprising one or more of the foregoing water swellable substances.

The core may optionally comprise an absorption enhancer. The absorption enhancer can be, for example, a fatty acid, a surfactant, a chelating agent, a bile salt, and combinations comprising one or more of the foregoing absorption enhancers. Specific examples of absorption enhancers are fatty acids such as capric acid, oleic acid and their monoglycerides, surfactants such as sodium lauryl sulfate, sodium taurocholate and polysorbate 80, chelating agents such as citric acid, phytic acid, ethylenediamine tetraacetic acid (EDTA) and ethylene glycol-bis(P-aminoethyl ether)-N,N,N,N-tetraacetic acid (EGTA). The core comprises 0 wt % to about 20 wt % of the absorption enhancer based on the total weight of the core, or about 2 wt % to about 10 wt % of the total weight of the core.

The lag time controlling layer is a semipermeable membrane comprising a water resistant polymer that is semipermeable for an aqueous solution, such as gastro-intestinal fluid. Suitable polymers are cellulose acetate, ethylcellulose, polyvinyl acetate, cellulose acetate butyrate, cellulose acetate propionate, acrylic acid copolymers, such as Eudragit® RS or RL, and combinations comprising one or more of the foregoing polymers. The polymer may optionally comprise pore forming agents, such as a water soluble substance, e.g. sucrose, salt; or a water soluble polymer e.g., polyethylene glycol. Also pharmaceutically acceptable excipients such as fillers and membrane strength influencing agents such as talc, aerosil, and/or sodium aluminum silicate may be included.

There may be at least one lag time controlling layer present in the dosage form. A lag time controlling layer positioned nearest the inner core material is constructed in the form of a semipermeable membrane that will disrupt after a desired time after ingestion. A desired lag time may be adjusted by the composition and thickness of the layer. The amount of substances forming such a disrupting semipermeable membrane, i.e., a lag time controlling layer, may be about 0.5 wt % to about 25 wt % of the weight of the core material including swelling substances or a swelling layer, or about 2 wt % to about 20 wt %.

The lag time controlling layer may comprise a mixture of ethylcellulose and talc. The mixture may contain 10% to 80% w/w (weight/weight) of talc.

Before applying the outer coating layer onto the layered pellets or tablets, they may optionally be covered with one or more separating layers comprising excipients. This separating layer separates the composition of the layered pellets or tablets from the outer enteric coating layer. Suitable materials for the optional separating layer are pharmaceutically acceptable compounds such as, for example, sugar, polyethylene glycol, polyvinyl pyrrolidone, polyvinyl alcohol, polyvinyl acetate, hydroxypropyl cellulose, methylcellulose, ethylcellulose, hydroxypropyl methylcellulose, carboxymethylcellulose sodium and others, and combinations comprising one or more of the foregoing materials. Other additives may also be included into the separating layer.

When the optional separating layer is applied to the layered pellets or tablets it may constitute a variable thickness. The maximum thickness of the optional separating layer is normally only limited by processing conditions. The separating layer may serve as a diffusion barrier and may act as a pH-buffering zone. The optional separating layer may improve the chemical stability of the active substance and/or the physical properties of the dosage form.

Finally the layered pellets or tablets are covered by one or more outer coating layers by using a suitable coating technique. The outer coating layer material may be dispersed or dissolved in either water or in suitable organic solvents. Suitable methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethyl ethylcellulose, shellac or other suitable coating layer polymer(s), and combinations comprising one or more of the foregoing polymers.

The applied polymer containing layers, and specially the outer coating layers may also contain pharmaceutically acceptable plasticizers to obtain desired mechanical properties.

Exemplary Formulations

The various release properties described above may be achieved in a variety of different ways. Suitable formulations include, for example, wax formulations, press coat formulations, easily administered formulations, osmotic pump dosage forms, etc.

Wax Formulations

A wax formulation is a solid dosage form comprising the paroxetine or a pharmaceutically acceptable salt thereof, most preferably paroxetine hydrochloride, in a waxy matrix. The waxy matrix may be prepared by hot melting a suitable wax material and using the melt to granulate the active agent material. The matrix material comprises the waxy material and the paroxetine.

The wax material can be, for example, an amorphous wax, an anionic wax, an anionic emulsifying wax, a bleached wax, a carnauba wax, a cetyl esters wax, a beeswax, a castor wax, a cationic emulsifying wax, a cetrimide emulsifying wax, an emulsifying wax, a glyceryl behenate, a microcrystalline wax, a nonionic wax, a nonionic emulsifying wax, a paraffin, a petroleum wax, a spermaceti wax, a white wax, a yellow wax, and combinations comprising one or more of the foregoing waxes. These and other suitable waxes are known to those of skill in the art. A cetyl esters wax, for example, may have a molecular weight of about 470 to about 490 and is a mixture containing primarily esters of saturated fatty alcohols and saturated fatty acids. The wax material can comprise a carnauba wax, glyceryl behenates, castor wax, and combinations comprising one or more of the foregoing waxes. When the waxy material consists of carnauba wax and no other waxy material is used, the matrix may be coated with a functional coating. When the waxy material includes glyceryl behenates and carnauba wax, the matrix can be used without a coating, but may have either a cosmetic coating or a functional coating depending on the precise release profile and appearance desired.

The wax material can be used at about 16 wt % to about 35 wt %, or about 20 wt % to about 32 wt %, or about 24 wt % to about 31 wt %, or about 28 wt % to about 29 wt % of the total weight of the matrix material. When a combination of wax is used, e.g., carnauba wax and glyceryl behenate, the component waxes can be used in a suitable ratio. Certain formulations include the wax material component from 100 to about 85 parts carnauba wax and from 0 to about 15 parts glyceryl behenate. In formulations that have a combination of carnauba wax and castor wax, for example, the wax component may have about 100 to about 85 parts carnauba wax and 0 to about 15 parts castor wax. When carnauba wax, glyceryl behenate and castor wax are present, the carnauba wax can comprise at least about 85 wt % of the waxy material and the balance of the waxy material is made up of a combination of glyceryl behenate and castor wax, in a suitable relative proportion.

Optionally, fatty acids and fatty acid soaps can be present in the waxy dosage form. In some cases, the fatty acids and/or fatty acid soaps can replace a portion of the wax or waxes. These optional fatty acids and fatty acid soaps can be those that are generally used in the pharmaceutical industry as tableting lubricants, such as, for example, solid fatty acids (for example fatty acids having about 16 to about 22 carbon atoms), and the alkaline earth metal salts thereof, particularly the magnesium and calcium salts, and combinations comprising one or more of the foregoing fatty acids. The fatty acid can be, for example, stearic acid. The optional fatty acids and fatty acid soaps, when present, can be used in amounts of up to about 10 wt % of the total weight of the matrix material, or about 2.5 wt % to about 9 wt %, or about 2.7 wt % to about 8.6 wt %, or about 3 wt % to about 6 wt % of the total weight of the matrix material. An amount of up to about 2 wt % of the total core formulation of the optional fatty acid materials may be used as a blend with the melt granulate. Amounts of at least about 1 wt % may be used in this manner with the remainder being added to the waxes for melting and granulating the active agent.

To prepare the dosage form, the waxes may be melted and used to granulate the paroxetine. The granulate may be allowed to cool and then be milled to a proper size. Advantageously, the granulate is milled to an average particle size of about 75 microns to about 850 microns, preferably about 150 microns to about 425 microns. The milled granulate may be mixed with optional processing aids. The processing aids include, for example, hydrophobic colloidal silicon dioxide (such as CAB-O-SIL® M5). Hydrophobic silicon dioxide may be used in amounts of less than or equal to about 0.5 wt %, but individual formulations can be varied as required. The blend of the waxy granulate and the processing aids, if any, may be compressed and then optionally coated.

The wax dosage form can include, for example, compressed coated or uncoated tablets, compressed pellets contained in capsules, or loose powder or powder filled capsules.

Press Coat Formulations

A press coat oral dosage form of paroxetine or a pharmaceutically acceptable salt thereof comprises a core composition and a coating composition press-coated on the core. The core composition comprises a waxy material and paroxetine or its salt, and the coating composition comprises a hydrophilic polymer and optionally paroxetine or its salt. The active agent may be in the form of paroxetine hydrochloride.

The core composition of the press coat dosage from comprises a waxy material. The waxy material can be a hydrophobic waxy material to provide controlled-release of the active agent. In pharmaceutical and/or veterinary products, for example, such waxy materials include, for example, carnauba wax, tribehenin, fatty alcohols (particularly those having 12-24 carbon atoms, such as lauryl alcohol, myristyl alcohol, stearyl alcohol, palmityl alcohol, etc.), fatty acids (particularly those having 12-24 carbon atoms, such as lauric acid, myristic acid, stearic acid, palmitic acid, etc), polyethylenes, castor wax, C16-30 fatty acid triglycerides, beeswax, and combinations comprising one or more of the foregoing waxes.

The coating composition comprises a hydrophilic polymer. The hydrophilic polymer can provide for controlled-release of the paroxetine. The hydrophilic polymer providing controlled-release may be a film forming polymer, such as a hydrophilic cellulose polymer. Such a hydrophilic cellulose polymer may be hydroxyalkyl cellulose polymer, for example hydroxyethyl cellulose (HEC), hydroxypropyl cellulose (HPC), hydroxypropyl methylcellulose (HPMC), hydroxypropyl ethylcellulose (HPEC), hydroxypropyl propylcellulose (HPPC), hydroxypropyl butylcellulose (HPBC), and combinations comprising one or more of the foregoing polymers.

Both the core composition and the coating composition may further include a filler, such as a water insoluble filler, water soluble filler, and mixtures thereof. A water-insoluble filler can be talc or a calcium salt such as a calcium phosphate, e.g., a dicalcium phosphate. The filler in the coating composition can be the same or different as the filler in the core composition, if any. For example, the core composition can include a water-soluble filler while the coating composition can include a water-insoluble filler.

Optional excipients can also be present in the core composition and the coating composition, including lubricants (such as talc and magnesium stearate), glidants (such as fumed or colloidal silica), pH modifiers (such as acids, bases and buffer systems), pharmaceutically useful processing aids, and combinations comprising one or more of the foregoing excipients. Excipients in the coating composition can be the same or different as those in the core composition.

In the formation of a dosage form, the core composition can be press-coated with the press-coat composition coating formulation to form a tablet. The tablet can be further coated with optional additional coatings. The additional coatings can be pH-dependent or pH-independent, aesthetic or functional, and can include the active agent in immediate or controlled-release. The optional additional coating can include an paroxetine, either active agent or a pharmaceutically active salt thereof or a different active agent than is contained in the core composition and the coating composition. The additional coating may, for example, include an immediate-release dosage form of paroxetine.

The press coat formulations may have substantially zero order, first order, and second order release rate profiles by adjusting the amount of paroxetine in the core composition and the coating composition. The ratio of the paroxetine in the core composition (CoreAA) to paroxetine in the coating composition (CoatAA) may be about 1:99 to about 99:1, or about 95:5 to about 5:99, or about 9:1 to about 1:9. For the highly soluble active agents, and other highly soluble active agents that may be used in combination with paroxetine, a CoreAA:CoatAA of about 3:4 to about 5:3 is can provide a substantially zero order release rate, a CoreAA:CoatAA of less than about 3:4 can provide a substantially first order release rate, and a CoreAA:CoatAA of greater than about 5:3 can provide a substantially second order release rate.

In forming the dosage form, the core composition components (paroxetine, wax, and optional excipients) are blended together and compressed into suitable cores. The blending can take place in a suitable order of addition. The cores may be blended by starting with the smallest volume component and then successively adding the larger volume components. Another process is to melt the wax and to blend the paroxetine and optional excipients into the melted wax. Alternatively, the paroxetine, wax and optional excipients can be blended together and then subjected to a temperature at which the wax will melt. Once cooled, the solidified mass can be milled into granules for compaction into cores.

The press coat formulations can be 12.5 mg, 25 mg, or 35 mg tablets press coated tablets. One exemplary press coat paroxetine formulation comprises 10 mg active agent in an immediate-release coating composition and 25 mg paroxetine between the core composition and the coating composition. In this example, the 0-4 hour cumulative release of paroxetine in 0.1 N hydrochloric acid is may be at least about 25 wt % to about 50 wt %, more preferably about 35 wt % to about 40 wt %, of the loaded dose, and the 0-12 hour cumulative release of the active agent in 0.1 N hydrochloric acid (simulated gastric fluid) may be at least about 75 wt %, more preferably at least about 85 wt %, of the dosage form dose. In another example, a 35 mg paroxetine formulation comprises a 3:2:1 (core:press coat:immediate-release coat) ratio, e.g., a core composition comprising 15 mg of paroxetine, a coating composition comprising 10 mg of paroxetine, and an immediate-release loading dose comprising 5 mg of paroxetine.

Easily Administered Dosage Forms

Chewable Tablets

Another solid dosage form is a chewable tablet containing paroxetine. A chewable tablet comprises a chewable base and optionally a sweetener. The chewable base comprises an excipient such as, for example, mannitol, sorbitol, lactose, or a combination comprising one or more of the foregoing excipients. The optional sweetener used in the chewable dosage form may be, for example, digestible sugars, sucrose, liquid glucose, sorbitol, dextrose, isomalt, liquid maltitol, aspartame, lactose, and combinations comprising one ore more of the foregoing sweeteners. In certain cases, the chewable base and the sweetener may be the same component. The chewable base and optional sweetener may comprise about 50 wt % to about 90 wt % of the total weight of the dosage form.

The chewable dosage form may additionally contain preservatives, agents that prevent adhesion to oral cavity and crystallization of sugars, flavoring agents, souring agents, coloring agents, and combinations comprising one or more of the foregoing agents. Glycerin, lecithin, hydrogenated palm oil or glyceryl monostearate may be used as a protecting agent of crystallization of the sugars in an amount of about 0.04 wt % to about 2.0 wt % of the total weight of the ingredients, to prevent adhesion to oral cavity and improve the soft property of the products. Additionally, isomalt or liquid maltitol may be used to enhance the chewing properties of the chewable dosage form.

A method of making a chewable dosage form of the active agent is similar to the method used to make soft confectionary. The method generally involves the formation of a digestible sugar blend to which is added a frappe mixture. The digestible sugar blend may be prepared from corn syrup and digestible sugars blended in parts by weight ratio of 90:10 to 10:90. This blend may be heated to temperatures above 250° F. to remove water and to form a molten mass. The frappe mixture may be prepared from gelatin, egg albumen, milk proteins such as casein, and vegetable proteins such as soy protein, and the like which are added to a gelatin solution and rapidly mixed at ambient temperature to form an aerated sponge like mass. The frappe mixture is then added to the molten candy base and mixed until homogenous at temperatures between 150° F. to about 250° F. A wax matrix containing the paroxetine may then be added as the temperature of the mix is lowered to about 120° F. to about 194° F., whereupon additional ingredients such as flavors, colorants, and preservatives may be added. The formulation is further cooled and formed to pieces of desired dimensions.

Fast Dissolving Formulations

Another oral dosage form is a non-chewable, fast dissolving dosage form of paroxetine. These dosage forms can be made by methods known to those of ordinary skill in the art of pharmaceutical formulations. For example, Cima Labs has produced oral dosage forms including microparticles and effervescents which rapidly disintegrate in the mouth and provide adequate taste-masking. Cima Labs has also produced a rapidly dissolving dosage form containing the active agent and a matrix that includes a nondirect compression filler and a lubricant. Zydis (ZYPREXA) is produced by Eli Lilly as in a rapidly dissolvable, freeze-dried, sugar matrix formulated as a rapidly dissolving tablet. U.S. Pat. No. 5,178,878 and U.S. Pat. No. 6,221,392 provide teachings regarding fast-dissolve dosage forms.

An exemplary fast dissolve dosage form includes a mixture incorporating a water and/or saliva activated effervescent disintegration agent and microparticles. The microparticles incorporate an active agent together with a protective material substantially encompassing the active agent. The term “substantially encompassing” as used in this context means that the protective material substantially shields the active agent from contact with the environment outside of the microparticle. Thus, each microparticle may incorporate a discrete mass of the active agent covered by a coating of the protective material, in which case the microparticle can be referred to as a “microcapsule”. Alternatively or additionally, each microparticle may have the active agent dispersed or dissolved in a matrix of the protective material. The mixture including the microparticles and effervescent agent desirably may be present as a tablet of a size and shape adapted for direct oral administration to a patient, such as a human patient. The tablet is substantially completely disintegrable upon exposure to water and/or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the microparticles are released and can be swallowed as a slurry or suspension of the microparticles. The microparticles thus may be transferred to the patient's stomach for dissolution in the digestive tract and systemic distribution of the pharmaceutical ingredient.

The term effervescent disintegration agent(s) includes compounds which evolve gas. The preferred effervescent agents evolve gas by means of chemical reactions which take place upon exposure of the effervescent disintegration agent to water and/or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water activated materials may be kept in a generally anhydrous state with little or no absorbed moisture or in a stable hydrated form since exposure to water will prematurely disintegrate the tablet. The acid sources or acid may be those which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations were intended to be dissolved in a glass of water. Acid anhydrides and acid of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, and combinations comprising one or more of the foregoing carbonates.

The effervescent disintegration agent is not always based upon a reaction which forms carbon dioxide. Reactants which evolve oxygen or other gasses which are pediatrically safe may be employed. Where the effervescent agent includes two mutually reactive components, such as an acid source and a carbonate source, it is preferred that both components react substantially completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, the amount of either acid or carbonate source may exceed the amount of the other component. This may be useful to enhance taste and/or performance of a tablet containing an overage of either component. In this case, it is acceptable that the additional amount of either component may remain unreacted.

In general, the amount of effervescent disintegration agent useful for the formation of tablets is about 5 wt % to about 50 wt % of the final composition, or about 15 wt % and about 30 wt %, or about 20 wt % to about 25 wt % of the total weight of the composition.

More specifically, the tablets should contain an amount of effervescent disintegration agent effective to aid in the rapid and complete disintegration of the tablet when orally administered. By “rapid”, it is understood that the tablets should disintegrate in the mouth of a patient in less than about 10 minutes, and desirably between about 30 seconds and about 7 minutes, or the tablet should dissolve in the mouth between about 30 seconds and about 5 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the microcapsules or other discrete inclusions.

The paroxetine may be present in microparticles. Each microparticle incorporates the paroxetine in conjunction with a protective material. The microparticle may be provided as a microcapsule or as a matrix-type microparticle. Microcapsules may incorporate a discrete mass of the paroxetine surrounded by a discrete, separately observable coating of the protective material. Conversely, in a matrix-type particle, the paroxetine is dissolved, suspended or otherwise dispersed throughout the protective material. Certain microparticles may include attributes of both microcapsules and matrix-type particle. For example, a microparticle may incorporate a core incorporating a dispersion of the paroxetine in a first protective material and a coating of a second protective material, which may be the same as or different from the first protective material surrounding the core. Alternatively, a microparticle may incorporate a core consisting essentially of the paroxetine and a coating incorporating the protective material, the coating itself having some of the paroxetine dispersed within it.

The microparticles may have about 75 to about 600 micron mean outside diameter, or about 150 to about 500 microns. Microparticles above about 200 microns may be used. Thus, the microparticles may be between about 200 mesh and about 30 mesh U.S. standard size, and more preferably between about 100 mesh and about 35 mesh.

Tablets can be manufactured by well-known tableting procedures. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion.

Another exemplary fast-dissolve dosage form is a hard, compressed, rapidly dissolvable dosage form adapted for direct oral dosing. The dosage form includes an active agent often in the form of a protected particle, and a matrix. The matrix includes a nondirect compression filler and a lubricant, although, it may include other ingredients as well. The dosage form is adapted to rapidly dissolve in the mouth of a patient, yet it has a friability of about 2 wt % or less when tested according to the U.S.P. Generally, the dosage form will also have a hardness of at least about 15 to 20 Newtons (1.5-2.0 kilopond (kp)). Not only does the dosage form dissolve quickly, it does so in a way that provides a positive organoleptic sensation to the patient. In particular, the dosage form dissolves with a minimum of unpleasant grit which is tactilely inconsistent with a positive organoleptic sensation to the patient.

The protective materials may include polymers conventionally utilized in the formation of microparticles, matrix-type microparticles and microcapsules. Among these are cellulosic materials such as naturally occurring cellulose and synthetic cellulose derivatives; acrylic polymers and vinyl polymers. Other simple polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trademark EUDRAGIT® by Rohm Pharma GmbH of Darmstadt, Germany.

Generally, when a coating is used, the coating may be used at greater than or equal to about 5 wt % of the weight of the resulting particles. The coating may constitute at least about 10 wt % of the particle. The upper limit of protective coating material used is generally less critical, except that where a rapid release of the active ingredient is desired, the amount of coating material should not be so great that the coating material impedes the release profile of the active agent when ingested. Thus, it may be possible to use greater than 100 percent of the weight of the core, thereby providing a relatively thick coating.

The filler may comprise a nondirect compression filler. Exemplary fillers include, for example, nondirect compression sugars and sugar alcohols. Such sugars and sugar alcohols include, without limitation, dextrose, mannitol, sorbitol, lactose and sucrose. Of course, dextrose, for example, can exist as either a direct compression sugar, i.e., a sugar which has been modified to increase its compressibility, or a nondirect compression sugar.

Generally, the balance of the formulation can be matrix. Thus the percentage of filler can approach 100 wt %. The amount of nondirect compression filler is about 25 wt % to about 95 wt %, or about 50 wt % and about 95 wt %, or about 60 wt % to about 95 wt % of the total weight of the formulation.

In the fast-dissolve dosage form, a relatively high proportion of lubricant may be used. Lubricants, and in particular, hydrophobic lubricants such as magnesium stearate, are generally used in an amount of about 0.25 wt % to about 5 wt % of the total weight of the formulation, according to the Handbook of Pharmaceutical Excipients. Specifically, the amount of lubricant used can be about 1 wt % to about 2.5 wt %, and more specifically about 1.5 wt % to about 2 wt %. Despite the use of this relatively high rate of lubricant, the formulations exhibit a superior compressibility, hardness, and rapid dissolution within the mouth.

Hydrophobic lubricants include, for example, alkaline stearates, stearic acid, mineral and vegetable oils, glyceryl behenate, sodium stearyl fumarate, and combinations comprising one or more of the foregoing lubricants. Hydrophilic lubricants can also be used.

The dosage forms may have a hardness of at least about 15 Newtons and are designed to dissolve spontaneously and rapidly in the mouth of a patient in less than about 90 seconds to thereby liberate the particles. The dosage form may dissolve in less than about 60 seconds, or less than about 45 seconds. This measure of hardness is based on the use of small tablets of less than about 0.25 inches in diameter. A hardness of at least about 20 Newtons may be employed for larger tablets. Direct compression techniques may be employed for the formation of the tablets.

Sprinkle Dosage Forms

Sprinkle dosage forms include particulate or pelletized forms of paroxetine, optionally having functional or non-functional coatings, with which a patient or a caregiver can sprinkle the particulate/pelletized dose into drink or onto soft food. A sprinkle dosage form may comprise particles of about 10 to about 100 micrometers in their major dimension. Sprinkle dosage forms may be in the form of optionally coated granules or as microcapsules. Sprinkle dosage forms may be immediate or controlled-release formulations such as sustained-release formulations. See U.S. Pat. No. 5,084,278, which is hereby incorporated by reference for its teachings regarding microcapsule formulations, which may be administered as sprinkle dosage forms.

Taste Masked Solid Dosage Forms

A solid taste masked dosage form comprises a core element comprising the paroxetine and a coating surrounding the core element. The core element comprising the paroxetine may be in the form of a capsule or be encapsulated by micro-encapsulation techniques, where a polymeric coating is applied to the formulation. The core element includes the paroxetine and may also include carriers or excipients, fillers, flavoring agents, stabilizing agents and/or colorants.

The taste masked dosage form may include about 77 wt % to about 100 wt %, or about 80 wt % to about 90 wt %, based on the total weight of the composition of the core element including the paroxetine; and about 20 wt % to about 70 wt %, of a substantially continuous coating on the core element formed from a coating material including a polymer. The core element includes about 52 wt % to about 85 wt % of the paroxetine; and approximately 5 wt % to about 25 wt % of a supplementary component selected from waxes, water insoluble polymers, enteric polymers, and partially water soluble polymers, other suitable pharmaceutical excipients, and combinations comprising one or more of the foregoing components.

The core element optionally includes carriers or excipients, fillers, flavoring agents, stabilizing agents, colorants, and combinations comprising one or more of the foregoing additives. Suitable fillers include, for example, insoluble materials such as silicon dioxide, titanium dioxide, talc, alumina, starch, kaolin, polacrilin potassium, powdered cellulose, and microcrystalline cellulose, and combinations comprising one or more of the foregoing fillers. Soluble fillers include, for example, mannitol, sucrose, lactose, dextrose, sodium chloride, sorbitol, and combinations comprising one or more of the foregoing fillers. The filler may be present in amounts of up to about 75 wt % based on the total weight of the composition. The particles of the core element may be in the range of the particle size set forth above for core particles of core elements.

The core element may be in the form of a powder, for example, having a particle size of about 35 μm to about 125 μm. The small particle size facilitates a substantially non-gritty feel in the mouth. Small particle sizes also minimize break-up of the particles in the mouth, e.g. by the teeth. When in the form of a powder, the taste masked dosage form may be administered directly into the mouth or mixed with a carrier such as water, or semi-liquid compositions such as yogurt, and the like. However, the taste masked active agent may be provided in any suitable unit dosage form.

The coating material of the taste-masked formulation may take a form which provides a substantially continuous coating and still provides taste masking. In some cases, the coating also provides controlled-release of the active agent. The polymer used in taste masked dosage form coating may be a water insoluble polymer such as, for example, ethyl cellulose. The coating material of the taste masked dosage form may further include a plasticizer.

A method of preparing taste-masked pharmaceutical formulations such as powdered formulations includes mixing a core element and a coating material in a diluent and spray drying the mixture to form a taste-masked formulation. Spray drying of the paroxetine and polymer in the solvent involves spraying a stream of air into an atomized suspension so that solvent is caused to evaporate leaving the active agent coated with the polymer coating material.

For a solvent such as methylene chloride, the solvent concentration in the drying chamber may be maintained above about 40,000 parts, or about 40,000 to about 100,000 parts per million of organic solvent. The spray-drying process for such solvents may be conducted at a process temperature of about SoC to about 35° C. Spray drying of the dosage forms may be undertaken utilizing either rotary, pneumatic or pressure atomizers located in either a co-current, counter-current or mixed-flow spray dryer or variations thereof. The drying gas may be heated or cooled to control the rate of drying. A temperature below the boiling point of the solvent may be used. Inlet temperatures may be about 40° C. to about 120° C. and outlet temperatures about 5° C. to about 35° C.

The coat formation may be optimized to meet the needs of the material or application. Controlling the process parameters including temperature, solvent concentration, spray dryer capacity, atomizing air pressure, droplet size, viscosity, total air pressure in the system and solvent system, allows the formation of a range of coats, ranging from dense, continuous, non-porous coats through to more porous microcapsule/polymer matrices.

A post-treatment step may be used to remove residual solvent. The post treatment may include a post drying step including drying the final product on a tray and drying the product at a bed temperature sufficient to remove excess solvent, but not degrade the active agent. Preferably the drying temperature is in the range of about 35° C. to about 4° C. Once completed, the product may be collected by a suitable method, such as collection by sock filters or cyclone collection.

Taste Masked Liquid Dosage Forms

Liquid dosage forms of the paroxetine may be formulated that also provide adequate taste masking. The taste-masked dosage forms may be liquid dosage forms such as those disclosed by F.H. Faulding, Inc. (U.S. Pat. No. 6,197,348). A taste masked liquid dosage form may comprise a suspension of microcapsules taste masked as a function of the pH of a suspending medium and a polymer coating. Many active agents are less soluble at higher or lower pH than at the pH value of the mouth, which is around 5.9. In these cases, the active agent can be insufficiently solubilized to be tasted if the equilibrium concentration is below the taste threshold. However, problems can arise if all of the suspended particles are not swallowed because the active agent which remains in the mouth is able to dissolve at the pH of the mouth. The use of polymeric coatings on the active agent particles, which inhibit or retard the rate of dissolution and solubilization of the active agent is one means of overcoming the taste problems with delivery of active agents in suspension. The polymeric coating allows time for all of the particles to be swallowed before the taste threshold concentration is reached in the mouth.

Optimal taste masked liquid formulations may be obtained when consideration is given to: (i) the pH of maximum insolubility of the active agent; (ii) the threshold concentration for taste of the active agent; (iii) the minimum buffer strength required in the medium to avoid delayed or after taste; (iv) the pH limit beyond which further increase or decrease of pH leads to unacceptable instability of the active agent; and (v) the compatibility and chemical, physical and microbial stability of the other ingredients to the pH values of the medium.

A taste masked liquid dosage form thus comprises the active agent, a polymer with a quaternary ammonium functionality encapsulating the active agent, and a suspending medium adjusted to a pH at which the active agent remains substantially insoluble, for suspending the encapsulated active agent. The active agent is taste masked by the combination of the polymer and suspending medium.

The paroxetine may be in the form of its neutral or salt form and may be in the form of particles, crystals, microcapsules, granules, microgranules, powders, pellets, amorphous solids or precipitates. The particles may further include other functional components. The paroxetine may have a defined particle size distribution, or in the region of about 0.1 μm to about 500 μm, or about 1 μm to about 250 μm, or about 10 μm to about 150 μm, where there is acceptable mouth feel and little chance of chewing on the residual particles and releasing the active agent to taste.

The taste masked liquid dosage form may include, along with the paroxetine, other functional components present for the purpose of modifying the physical, chemical, or taste properties of the paroxetine. For example the paroxetine may be in the form of ion-exchange or cyclodextrin complexes or the paroxetine may be included as a mixture or dispersion with various additives such as waxes, lipids, dissolution inhibitors, taste-masking or -suppressing agents, carriers or excipients, fillers, and combinations comprising one or more of the foregoing components.

The polymer used to encapsulate the paroxetine or the pharmaceutical unit may be a polymer having a quaternary ammonium functionality, i.e., a polymer having quaternary ammonium groups on the polymer backbone. These polymers are effective in preventing the taste perception of the paroxetine when the resulting microcapsules are formulated as suspensions and stored for long periods despite their widely recognized properties of being permeable to water and dissolved active agents. A suitable polymer is a copolymer of acrylic and methacrylic acid esters with quaternary ammonium groups. The polymer may be a copolymer of methyl methacrylate and triethylammonium methacrylate. Specific examples of suitable polymer include EUDRAGIT RS or EUDRAGIT RL, available from Rohm America, LLC, Piscataway, N.J. used individually or in combination to change the permeability of the coat. A polymer coat having a blend of the RS or RL polymer along with other pharmaceutically acceptable polymers may also be used. These other polymers may be cellulose ethers such as ethyl cellulose, cellulose esters such as cellulose acetate and cellulose propionate, polymers that dissolve at acidic or alkaline pH, such as EUDRAGIT E, cellulose acetate phthalate, and hydroxypropylmethyl cellulose phthalate.

The quantity of polymer used in relation to the active agent is about 0.01-10:1, or about 0.02-1:1, or about 0.03-0.5:1, or about 0.05-0.3:1 by weight.

The pharmaceutically active agent or the active agent particle may be suspended, dispersed or emulsified in the suspending medium after encapsulation with the polymer. The suspending medium may be a water-based medium, but may be a non-aqueous carrier as well, constituted at an optimum pH for the active agent or pharmaceutical unit, such that the active agent remains substantially insoluble. The pH and ionic strength of the medium may be selected on the basis of stability, solubility and taste threshold to provide the optimum taste masking effect, and which is compatible with the stability of the active agent the polymer coat and the coating excipients.

Buffering agents may be included in the suspending medium for maintaining the desired pH. The buffering agents may include dihydrogen phosphate, hydrogen phosphate, amino acids, citrate, acetate, phthalate, tartrate salts of the alkali or alkaline earth metal cations such as sodium, potassium, magnesium, calcium, and combinations comprising one or more of the foregoing buffering agents. The buffering agents may be used in a suitable combination for achieving the required pH and may be of a buffer strength of about 0.01 to about 1 moles/liter of the final formulation, or about 0.01 to about 0.1 moles/liter, or about 0.02 to about 0.05 moles/liter.

The taste masked liquid dosage form may further include other optional dissolved or suspended agents to provide stability to the suspension. These include suspending agents or stabilizers such as, for example, methyl cellulose, sodium alginate, xanthan gum, (poly)vinyl alcohol, microcrystalline cellulose, colloidal silicas, bentonite clay, and combinations comprising one or more of the foregoing agents. Other agents used include preservatives such as methyl, ethyl, propyl and butyl parabens, sweeteners such as sucrose, saccharin sodium, aspartame, mannitol, flavorings such as grape, cherry, peppermint, menthol and vanilla flavors, and antioxidants or other stabilizers, and combinations comprising one or more of the foregoing agents.

A method of preparing a taste masked dosage form for oral delivery comprises encapsulating the paroxetine with a polymer such as one having a quaternary ammonium functionality; and adding a suspending medium adjusted to a pH at which the paroxetine is substantially insoluble, for suspending the encapsulated paroxetine; wherein the paroxetine is taste masked by the combination of the polymer and the medium. In the process, the polymer for encapsulation of the paroxetine or paroxetine-containing particle is dissolved in a solution or solvent chosen for its poor solubility for the paroxetine and good solubility for the polymer. Examples of appropriate solvents include but are not limited to methanol, ethanol, isopropanol, chloroform, methylene chloride, cyclohexane, and toluene, either used in combination or used alone. Aqueous dispersions of polymers may also be used for forming the paroxetine microparticles.

Encapsulation of the paroxetine or pharmaceutical unit by the polymer may be performed by a method such as suspending, dissolving, or dispersing the paroxetine in a solution or dispersion of polymer coating material and spray drying, fluid-bed coating, simple or complex coacervation, coevaporation, co-grinding, melt dispersion and emulsion-solvent evaporation techniques, and the like.

The polymer coated paroxetine powder can also as an alternative be applied for the preparation of reconstitutable powders, i.e.; dry powder active agent products that are reconstituted as suspensions in a liquid vehicle such as water before usage. The reconstitutable powders have a long shelf life and the suspensions, once reconstituted, have adequate taste masking.

Osmotic Pump Dosage Forms

Another dosage form of the paroxetine is one formulated with OROS technology (Alza Corporation, Mountain View, Calif.) also know as an “osmotic pump”. Such dosage forms have a fluid-permeable (semipermeable) membrane wall, an osmotically active expandable driving member (the osmotic push layer), and a density element for delivering the paroxetine. In an osmotic pump dosage form, the paroxetine may be dispensed through an exit means comprising a passageway, orifice, or the like, by the action of the osmotically active driving member. The paroxetine of the osmotic pump dosage form may be formulated as a thermo-responsive formulation in which the paroxetine is dispersed in a thermo-responsive composition. Alternatively, the osmotic pump dosage form may contain a thermo-responsive element comprising a thermo-responsive composition at the interface of the osmotic push layer and the paroxetine composition.

The osmotic pump dosage form comprises a semipermeable membrane. The capsule or other dispenser of the osmotic pump dosage form can be provided with an outer wall comprising the selectively semipermeable material. A selectively permeable material is one that does not adversely affect a host or animal, is permeable to the passage of an external aqueous fluid, such as water or biological fluids, while remaining essentially impermeable to the passage of the paroxetine, and maintains its integrity in the presence of a thermotropic thermo-responsive composition, that is it does not melt or erode in its presence. The selectively semipermeable material forming the outer wall is substantially insoluble in body fluids, nontoxic, and non-erodible.

Representative materials for forming the selectively semipermeable wall include semipermeable homopolymers, semipermeable copolymers, and the like. Suitable materials include, for example, cellulose esters, cellulose monoesters, cellulose diesters, cellulose triesters, cellulose ethers, cellulose ester-ethers, and combinations comprising one or more of the foregoing materials. These cellulosic polymers have a degree of substitution, D.S., on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, aroyl, alkyl, alkenyl, alkoxy, halogen, carboalkyl, alkylcarbamate, alkylcarbonate, alkylsulfonate, alkylsulfamate, and like semipermeable polymer forming groups.

Other selectively semipermeable materials include, for example, cellulose acylate, cellulose diacylate, cellulose triacylate, cellulose acetate, cellulose diacetate, cellulose triacetate, mono-, di- and tri-cellulose alkanylates, mono-, di- and tri-alkenylates, mono-, di- and tri-aroylates, and the like, and combinations comprising one or more of the foregoing materials. Exemplary polymers including cellulose acetate having a D.S. of 1.8 to 2.3 and an acetyl content of about 32 to about 39.9%; cellulose diacetate having a D.S. of 1 to 2 and an acetyl content of about 21 to about 35%; cellulose triacetate having a D.S of 2 to 3 and an acetyl content of about 34 to about 44.8%, and the like. More specific cellulosic polymers include cellulose propionate having a D.S. of 1.8 and a propionyl content of about 38.5%; cellulose acetate propionate having an acetyl content of about 1.5 to about 7% and an propionyl content of about 39 to about 42%; cellulose acetate propionate having an acetyl content of about 2.5 to about 3%, an average propionyl content of about 39.2 to about 45% and a hydroxyl content of about 2.8 to about 5.4%; cellulose acetate butyrate having a D.S. of 1.8, an acetyl content of about 13 to about 15%, and a butyryl content of about 34 to about 39%; cellulose acetate butyrate having an acetyl content of about 2 to about 29.5%, a butyryl content of about 17 to about 53%, and a hydroxyl content of about 0.5 to about 4.7%; cellulose triacylates having a D.S. of 2.9 to 3 such as cellulose trivalerate, cellulose trilaurate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate and the like; mixed cellulose esters such as cellulose acetate valerate, cellulose acetate succinate, cellulose propionate succinate, cellulose acetate octanoate, cellulose valerate palmitate, cellulose acetate heptonate, and the like, and combinations comprising one or more of the foregoing polymers.

Additional selectively semipermeable polymers include, for example, acetaldehyde dimethyl cellulose acetate, cellulose acetate ethylcarbamate, cellulose acetate methylcarbamate, cellulose dimethylaminoacetate, semi-permeable polyamides, semipermeable polyurethanes, semi-permeable polysulfanes, semipermeable sulfonated polystyrenes, cross-linked, selectively semipermeable polymers formed by the coprecipitation of a polyanion and a polycation, selectively semipermeable silicon rubbers, semipermeable polystyrene derivates, semipermeable poly(sodium styrenesulfonate), semipermeable poly(vinylbenzyltrimethyl) ammonium chloride polymers, and combinations comprising one or more of the foregoing polymers.

The osmotically expandable driving member, or osmotic push layer, of the osmotic pump dosage form is swellable and expandable inner layer. The materials used for forming the osmotic push layer, are neat polymeric materials, and/or polymeric materials blended with osmotic agents that interact with water or a biological fluid, absorb the fluid, and swell or expand to an equilibrium state. The polymer should exhibit the ability to retain a significant fraction of imbibed fluid in the polymer molecular structure. Such polymers may be, for example, gel polymers that can swell or expand to a very high degree, usually exhibiting about a 2 to 50-fold volume increase. Swellable, hydrophilic polymers, also known as osmopolymers, can be non-cross-linked or lightly cross-linked. The cross-links can be covalent or ionic bonds with the polymer possessing the ability to swell but not dissolve in the presence of fluid. The polymer can be of plant, animal or synthetic origin. Polymeric materials useful for the present purpose include poly(hydroxyalkyl methacrylate) having a molecular weight of about 5,000 to about 5,000,000, poly(vinylpyrrolidone) having a molecular weight of about 10,000 to about 360,000, anionic and cationic hydrogels, poly(electrolyte) complexes, poly(vinyl alcohol) having a low acetate residual, a swellable mixture of agar and carboxymethyl cellulose, a swellable composition comprising methyl cellulose mixed with a sparingly crosslinked agar, a water-swellable copolymer produced by a dispersion of finely divided copolymer of maleic anhydride with styrene, ethylene, propylene, or isobutylene, water swellable polymer of N-vinyl lactams, and the like, and combinations comprising one or more of the foregoing polymers. Other gelable, fluid imbibing and retaining polymers useful for forming the osmotic push layer include pectin having a molecular weight ranging of about 30,000 to about 300,000, polysaccharides such as agar, acacia, karaya, tragacanth, algins and guar, acidic carboxy polymer and its salt derivatives, polyacrylamides, water-swellable indene maleic anhydride polymers; polyacrylic acid having a molecular weight of about 80,000 to about 200,000; POLYOX, polyethylene oxide polymers having a molecular weight of about 100,000 to about 5,000,000, and greater, starch graft copolymers, polyanions and polycations exchange polymers, starch-polyacrylonitrile copolymers, acrylate polymers with water absorbability of about 400 times its original weight, diesters of polyglucan, a mixture of cross-linked polyvinyl alcohol and poly(N-vinyl-2-pyrrolidone), zein available as prolamine, poly(ethylene glycol) having a molecular weight of about 4,000 to about 100,000, and the like, and combinations comprising one or more of the foregoing polymers.

The osmotically expandable driving layer of the osmotic pump dosage fomm may further contain an osmotically effective compound (osmagent) that can be used neat or blended homogeneously or heterogeneously with the swellable polymer, to form the osmotically expandable driving layer. Such osmagents include osmotically effective solutes that are soluble in fluid imbibed into the swellable polymer, and exhibit an osmotic pressure gradient across the semipermeable wall against an exterior fluid. Suitable osmagents include, for example, solid compounds such as magnesium sulfate, magnesium chloride, sodium chloride, lithium chloride, potassium sulfate, sodium sulfate, mannitol, urea, sorbitol, inositol, sucrose, glucose, and the like, and combinations comprising one or more of the foregoing osmagents. The osmotic pressure in atmospheres, atm, of the osmagents may be greater than about zero atm, and generally about zero atm to about 500 atm, or higher.

The swellable, expandable polymer of the osmotically expandable driving layer, in addition to providing a driving source for delivering the active agent from the dosage form, may also function as a supporting matrix for an osmotically effective compound. The osmotic compound can be homogeneously or heterogeneously blended with the polymer to yield the desired expandable wall or expandable pocket. The composition in a presently preferred embodiment comprises (a) at least one polymer and at least one osmotic compound, or (b) at least one solid osmotic compound. Generally, a composition will comprise about 20 wt % to about 90 wt % of polymer and about 10 wt % to about 80 wt % of osmotic compound. In one embodiment, the composition comprises about 35 wt % to about 75 wt % of polymer and about 25 wt % to about 65 wt % of osmotic compound, based on the total weight of the composition.

The paroxetine of the osmotic pump dosage form may be formulated as a thermo-responsive formulation in which the paroxetine is dispersed in a thermo-responsive composition. Alternatively, the osmotic pump dosage form may contain a thermo-responsive element comprising a thermo-responsive composition at the interface of the osmotic push layer and the paroxetine composition. Representative thermo-responsive compositions and their melting points are as follows: Cocoa butter (32° C.-34° C.), cocoa butter plus 2% beeswax (35° C.-37° C.), propylene glycol monostearate and distearate (32° C.-35° C.), hydrogenated oils such as hydrogenated vegetable oil (36° C.-37.5° C.), 80% hydrogenated vegetable oil and 20% sorbitan monopalmitate (39° C.-39.5° C.), 80% hydrogenated vegetable oil and 20% polysorbate 60, (36° C.-37° C.), 77.5% hydrogenated vegetable oil, 20% sorbitan trioleate, 2.5% beeswax and 5.0% distilled water, (37° C.-38° C.), mono-, di-, and triglycerides of acids having from 8-22 carbon atoms including saturated and unsaturated acids such as palmitic, stearic, oleic, lineolic, linolenic and archidonic; triglycerides of saturated fatty acids with mono- and diglycerides (34° C.-35.5° C.), propylene glycol mono- and distearates 3(33° C.-34° C.), partially hydrogenated cottonseed oil (35° C.-39° C.), a block polymer of polyoxy-alkylene and propylene glycol; block polymers comprising 1,2-butylene oxide to which is added ethylene oxide; block copolymers of propylene oxide and ethylene oxide, hardened fatty alcohols and fats (33° C.-36° C.), hexadienol and hydrous lanolin triethanolamine glyceryl monostearate (38° C.), eutectic mixtures of mono-, di-, and triglycerides (35° C.-39° C.), WITEPSOL#15, triglyceride of saturated vegetable fatty acid with monoglycerides (33.5° C.-35.5° C.), WITEPSOL H32 free of hydroxyl groups (31° C.-33° C.), WITEPSOL W25 having a saponification value of 225-240 and a melting point of (33.5° C.-35.5° C.), WITEPSOL E75 having a saponification value of 220-230 and a melting point of (37° C.-39° C.), a polyalkylene glycol such as polyethylene glycol 1000, a linear polymer of ethylene oxide (38° C.-41° C.), polyethylene glycol 1500 (38° C.-41° C.), polyethylene glycol monostearate (39° C.-42.5° C.), 33% polyethylene glycol 1500, 47% polyethylene glycol 6000 and 20% distilled water (39° C.-41° C.), 30% polyethylene glycol 1500, 40% polyethylene glycol 4000 and 30% polyethylene glycol 400, (33° C.-38° C.), mixture of mono-, di-, and triglycerides of saturated fatty acids having 11 to 17 carbon atoms, (33° C.-35° C.), and the like. The thermo-responsive compositions, including thermo-responsive carriers are useful for storing the active agent in a solid composition at a temperature of about 20° C. to about 33° C., maintaining an immiscible boundary at the swelling composition interface, and for dispensing the agent in a flowable composition at a temperature greater than about 33° C. and preferably between about 33° C. and about 40° C.

The amount of paroxetine present in the osmotic pump dosage form is about 25 mg to about 2 g or more. The osmotic dosage form may be formulated for once daily or less frequent administration.

The paroxetine of the osmotic pump dosage form may be formulated by a number of techniques known in the art for formulating solid and liquid oral dosage forms. The paroxetine of the osmotic pump dosage form may be formulated by wet granulation. In an exemplary wet granulation method, the paroxetine and the ingredients comprising the paroxetine layer are blended using an organic solvent, such as isopropyl alcohol-ethylene dichloride 80:20 v:v (volume:volume) as the granulation fluid. Other granulating fluid such as denatured alcohol 100% may be used for this purpose. The ingredients forming the paroxetine layer are individually passed through a screen such as a 40-mesh screen and then thoroughly blended in a mixer. Next, other ingredients comprising the active agent layer are dissolved in a portion of the granulation fluid, such as the cosolvent described above. Then the latter prepared wet blend is slowly added to the active agent blend with continual mixing in the blender. The granulating fluid is added until a wet blend is produced, which wet mass then is forced through a screen such as a 20-mesh screen onto oven trays. The blend is dried for about 18 to about 24 hours at about 30° C. to about 50° C. The dry granules are sized then with a screen such as a 20-mesh screen. Next, a lubricant is passed through a screen such as an 80-mesh screen and added to the dry screen granule blend. The granulation is put into milling jars and mixed on ajar mill for about 1 to about 15 minutes. The push layer may also be made by the same wet granulation techniques. The compositions are pressed into their individual layers in a KILIAN press-layer press.

Another manufacturing process that can be used for providing the paroxetine layer and osmotically expandable driving layer comprises blending the powered ingredients for each layer independently in a fluid bed granulator. After the powered ingredients are dry blended in the granulator, a granulating fluid, for example, poly(vinyl-pyrrolidone) in water, or in denatured alcohol, or in 95:5 ethyl alcohol/water, or in blends of ethanol and water is sprayed onto the powders. Optionally, the ingredients can be dissolved or suspended in the granulating fluid. The coated powders are then dried in a granulator. This process granulates the ingredients present therein while adding the granulating fluid. After the granules are dried, a lubricant such as stearic acid or magnesium stearate is added to the granulator. The granules for each separate layer are pressed then in the manner described above.

The paroxetine formulation and osmotic push layer of the osmotic dosage form may also be manufactured by mixing an paroxetine with composition forming ingredients and pressing the composition into a solid lamina possessing dimensions that correspond to the internal dimensions of the compartment. In another manufacture, the paroxetine and other paroxetine composition-forming ingredients and a solvent are mixed into a solid, or a semisolid, by methods such as ballmilling, calendaring, stirring or rollmilling, and then pressed into a preselected layer forming shape. Next, a layer of a composition comprising an osmopolymer and an optional osmagent are placed in contact with the layer comprising the paroxetine. The layering of the first layer comprising the paroxetine and the second layer comprising the osmopolymer and optional osmagent composition can be accomplished by using a conventional layer press technique. The semipermeable wall can be applied by molding, spraying or dipping the pressed bilayer's shapes into wall forming materials. An air suspension coating procedure which includes suspending and tumbling the two layers in current of air until the wall forming composition surrounds the layers is also used to form the semi-permeable wall of the osmotic dosage forms.

The dispenser of the osmotic pump dosage form may be in the form of a capsule. The capsule may comprise an osmotic hard capsule and/or an osmotic soft capsule. The osmotic hard capsule may be composed of two parts, a cap and a body, which are fitted together after the larger body is filled with the active agent. The osmotic hard capsule may be fitted together by slipping or telescoping the cap section over the body section, thus completely surrounding and encapsulating the active agent. Hard capsules may be made by techniques known in the art.

The soft capsule of the osmotic pump dosage form may be a one-piece osmotic soft capsule. Generally, the osmotic soft capsule is of sealed construction encapsulating the active agent. The soft capsule may be made by various processes, such as the plate process, the rotary die process, the reciprocating die process, and the continuous process.

Materials useful for forming the capsule of the osmotic pump dosage form are commercially available materials including gelatin, gelatin having a viscosity of about 5 to about 30 millipoises and a bloom strength up to about 150 grams; gelatin having a bloom value of about 160 to about 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia, glycerin, and water; and the like, and combinations comprising one or more of the foregoing materials.

The semipermeable wall forming composition can be applied to the exterior surface of the capsule in laminar arrangement by molding, forming, air spraying, dipping or brushing with a semipermeable wall forming composition. Other techniques that can be used for applying the semipermeable wall are the air suspension procedure and the pan coating procedures. The air suspension procedure includes suspending and tumbling the capsule arrangement in a current of air and a semipermeable wall forming composition until the wall surrounds and coats the capsule. The procedure can be repeated with a different semipermeable wall forming composition to form a semipermeable laminated wall.

Exemplary solvents suitable for manufacturing the semipermeable wall include inert inorganic and organic solvents that do not adversely harm the materials, the capsule wall, the active agent, the thermo-responsive composition, the expandable member, or the final dispenser. Solvents for manufacturing the semipermeable wall may be aqueous solvents, alcohols, ketones, esters, ethers, aliphatic hydrocarbons, halogenated solvents, cycloaliphatics, aromatics, heterocyclic solvents, and combinations comprising one or more of the foregoing solvents. Particular solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride, nitroethane, nitropropane, tetrachloroethane, ethyl ether, isopropyl ether, cyclohexane, cyclooctane, benzene, toluene, naphtha, 1,4-dioxane, tetrahydrofuran, water, and mixtures thereof such as acetone and water, acetone and methanol, acetone and ethyl alcohol, methylene dichloride and methanol, and ethylene dichloride, methanol, and combinations comprising one or more of the foregoing solvents. The semipermeable wall may be applied at a temperature a few degrees less than the melting point of the thermo-responsive composition. Alternatively, the thermo-responsive composition can be loaded into the dispenser after applying the semipermeable wall.

The exit means or hole in the osmotic pump dosage form, for releasing the active agent, can be formed by mechanical or laser drilling, or by eroding an erodible element in the wall, such as a gelatin plug. The orifice can be a polymer inserted into the semipermeable wall, which polymer is a porous polymer and has at least one pore, or which polymer is a microporous polymer and has at least one micro-pore.

Solid State Dispersions

Another dosage form is a solid state dispersion. A “solid state dispersion” is a dispersion of one or more active agents in an inert carrier or matrix in a solid state prepared by a melting (fusion), solvent, or combined melt-solvent method. The dispersion of an active agent in a solid carrier or diluent by traditional mechanical mixing is not included within the definition of this term. Solid state dispersions are particularly advantageous for use with poorly soluble drugs, such as paroxetine.

Suitable carriers include, for example, hydroxypropyl cellulose, methyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polypropylene, dextrans, dextrins, hydroxypropyl-beta-cyclodextrin, chitosan, co(lactic/glycolid) copolymers, poly(orthoester), poly(anhydrate), polyvinyl chloride, polyvinyl acetate, ethylene vinyl acetate, lectins, carbopols, silicon elastomers, polyacrylic polymers, maltodextrins, lactose, fructose, inositol, trehalose, maltose, raffinose, polyvinylpyrrolidone (PVP), polyethylene glycol (PEG), and alpha-, beta-, and gamma-cyclodextrins, crospovidone, and combinations comprising one or more of the foregoing carriers.

Suitable methods for forming solid state dispersions include, for example, the “solvent method”, in which the active agent is dispersed in a water soluble carrier by dissolving a physical mixture containing the active agent and the pharmaceutically acceptable carrier in a common organic solvent and then removing the solvent by evaporation. The resulting solid dispersion is recovered and used in the preparation of suitable pharmaceutical compositions. Manufacture of solid dispersions by the fusion or “melt” process involves combination of the pharmaceutically acceptable carrier and the poorly water soluble drug where the two components are allowed to melt at temperatures at or above the melting point of both the active agent and the carrier. In the fusion process, the drug and carrier are first physically mixed and then both are melted. The molten mixture is then cooled rapidly to provide a congealed mass which is subsequently milled to produce a powder.

Another method for forming a solid dispersion comprises a solvent process comprising forming a solution comprising a carrier and a non-aqueous solvent. Suitable non-aqueous solvents include, for example, an alcohol selected from methanol, ethanol, n-propanol, iso-propanol, n-butanol, iso-butanol, and sec-butanol, and combinations comprising one or more of the foregoing solvents. The non-aqueous solvent may be dry or anhydrous. In forming a solution of a polymeric carrier and a non-aqueous solvent, it is understood that heating of the solution is allowable, but is not required, provided that the temperature does not result in decomposition or degradation of any materials.

Upon forming the solution, the process proceeds by dissolving the free base of a poorly water soluble active agent in the solution thus formed. Heating is allowed, but not required. Addition of a poorly soluble active agent is not limited to one active agent but might encompass a combination of one or more active agents provided at least one active agent is a poorly water soluble active agent in the form of a free base. The ratio by weight of carrier to poorly soluble active agent can be about 5:1 to about 1:1; or about 4:1 to about 1:1; or about 3:1 to about 1.5:1; or about 2:1. The order of addition for the polymeric carrier, the nonaqueous solvent and the free base of the poorly water soluble active agent is interchangeable. For example, the free base active agent could be dissolved into the non-aqueous solvent after which the polymeric carrier is added.

Upon dissolution of the free base active agent, the process proceeds converting the free base of the active agent to a pharmaceutically acceptable salt. The salt can be formed by addition of an inorganic or an organic acid which preferably is non-toxic and pharmaceutically acceptable. The acid may be added either as a gas, a liquid or as a solid dissolved into a nonaqueous solvent. The acid may be dry hydrogen chloride and the molar quantity of acid added to the solution of the active agent free base and carrier may either be in stoichiometric proportion to the active agent free base or be in excess of the molar quantity of the active agent free base, especially when added as a gas. Upon addition of the acid, the formed free base salt remains dissolved in solution with the polymeric carrier.

Lastly, upon formation of the free base salt, the process proceeds by recovering the non-aqueous solvent to form a solid state dispersion of the free base salt in the polymeric carrier. A method of removal of the non-aqueous solvent which renders a substantially homogeneous solid state dispersion is intended. Suitable methods of evaporation under vacuum include rotoevaporation, static vacuum drying, and a combination thereof. One skilled in the art of pharmaceutical formulations can determine a reasonable temperature at which the non-aqueous solvent can be removed, provided the temperature is not so high as to cause degradation or decomposition of the materials; however, such as about 20° C. to about 50° C. Evaporation of the non-aqueous solvent should render a solid state dispersion which is homogeneous and substantially free of non-aqueous solvent. By substantially free it is meant that the solid state dispersion contains less than about 20 wt % of residual non-aqueous solvent, or less than about 10 wt %, or less than about 5 wt %, or less than about 1 wt %, based on the total weight of the dispersion.

The ratio of active agent free base to the pharmaceutically acceptable carrier can be varied over a wide range and depends on the concentration of active agent required in the pharmaceutical dosage form ultimately administered. However, the preferred range of paroxetine in the solid dispersion is about 16 wt % to about 50 wt % of the total solid dispersion weight, or about 20 wt % to about 50 wt %, or about 25 wt % to about 40 wt %, or about 33 wt % of the total dispersion weight.

Alternatively, the general method for preparation of a solid dispersion can proceed by a fusion process wherein a carrier is mixed with a poorly water soluble drug, or drug combination, to form an intimate mixture. The mixture is heated at or near the temperature of the highest melting point of either the pharmaceutically acceptable carrier or poorly water soluble drug or drug combination, thus forming a melt. The polymeric carrier may be polyethylene glycol. A preferred ratio by weight of water soluble pharmaceutically acceptable polymeric carrier to poorly water soluble drug about 5:1 to about 1:1; or about 4:1 to about 1:1; or about 3:1 to about 1.5:1; or about 2:1.

Upon forming the molten homogeneous melt, the process proceeds by diffusing dry hydrogen chloride gas through the molten drug/carrier mixture to effect salt formation of the drug. Lastly, upon formation of the free base salt, the process proceeds by cooling the molten homogeneous melt by conventional methods to form a water soluble solid state dispersion.

Controlled-Release Formulation for Release into the Stomach and Upper Gastrointestinal Tract

An exemplary controlled-release formulation is one in which the paroxetine is dispersed in a polymeric matrix that is water-swellable rather than merely hydrophilic, that has an erosion rate that is substantially slower than its swelling rate, and that releases the paroxetine primarily by diffusion. The rate of diffusion of the paroxetine out of the matrix can be slowed by increasing the paroxetine particle size, by the choice of polymer used in the matrix, and/or by the choice of molecular weight of the polymer. The matrix is a relatively high molecular weight polymer that swells upon ingestion, preferably to a size that is at least about twice its unswelled volume, and that promotes gastric retention during the fed mode. Upon swelling, the matrix may also convert over a prolonged period of time from a glassy polymer to a polymer that is rubbery in consistency, or from a crystalline polymer to a rubbery one. The penetrating fluid then causes release of the paroxetine in a gradual and prolonged manner by the process of solution diffusion, i.e., dissolution of the paroxetine in the penetrating fluid and diffusion of the dissolved drug back out of the matrix. The matrix itself is solid prior to administration and, once administered, remains undissolved in (i.e., is not eroded by) the gastric fluid for a period of time sufficient to permit substantially all of the active agent to be released by the solution diffusion process during the fed mode. By substantially all, it is meant greater than or equal to about 90 wt %, preferably greater than or equal to about 95 wt % of the paroxetine or pharmaceutically acceptable salt thereof is released. The rate-limiting factor in the release of the paroxetine may be therefore controlled diffusion of the paroxetine from the matrix rather than erosion, dissolving, or chemical decomposition of the matrix.

For highly soluble active agents, the swelling of the polymeric matrix thus achieves two objectives—(i) the tablet swells to a size large enough to cause it to be retained in the stomach during the fed mode, and (ii) it retards the rate of diffusion of the highly soluble active agent long enough to provide multi-hour, controlled delivery of the active agent into the stomach. The water-swellable polymer forming the matrix is a polymer that is non-toxic, that swells in a dimensionally unrestricted manner upon imbibition of water, and that provides for sustained-release of an incorporated active agent. Examples of suitable polymers include, for example, cellulose polymers and their derivatives (such as for example, hydroxyethylcellulose, hydroxypropylcellulose, carboxymethylcellulose, and microcrystalline cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly (2-ethyl-2-oxazoline), poly(ethyleneimine), polyurethane hydrogels, and crosslinked polyacrylic acids and their derivatives. Further examples are copolymers of the polymers listed in the preceding sentence, including block copolymers and grafted polymers. Specific examples of copolymers are PLURONIC® and TECTONIC®, which are polyethylene oxide-polypropylene oxide block copolymers available from BASF Corporation, Chemicals Div., Wyandotte, Mich., USA.

The terms “cellulose” and “cellulosic” denote a linear polymer of anhydroglucose. Cellulosic polymers include, for example, alkyl-substituted cellulosic polymers that ultimately dissolve in the gastrointestinal (GI) tract in a predictably delayed manner. Alkyl-substituted cellulose derivatives may be those substituted with alkyl groups of 1 to 3 carbon atoms each. Specific examples are methylcellulose, hydroxymethylcellulose, hydroxyethylcellulose, hydroxypropylcellulose, hydroxypropyl methylcellulose, and carboxymethylcellulose. In terms of their viscosities, one class of suitable alkyl-substituted celluloses includes those whose viscosity is about 100 to about 110,000 centipoise as a 2% aqueous solution at 20° C. Another class includes those whose viscosity is about 1,000 to about 4,000 centipoise as a 1% aqueous solution at 20° C. Exemplary alkyl-substituted celluloses are hydroxyethyl cellulose and hydroxypropyl methylcellulose. A specific example of a hydroxyethylcellulose is NATRASOL® 250HX NF (National Formulary), available from Aqualon Company, Wilmington, Del., USA.

Suitable polyalkylene oxides are those having the properties described above for alkyl-substituted cellulose polymers. An example of a polyalkylene oxide is poly(ethylene oxide), which term is used herein to denote a linear polymer of unsubstituted ethylene oxide. Poly(ethylene oxide) polymers having molecular weights of about 4,000,000 and higher are preferred. More preferred are those with molecular weights of about 4,500,000 to about 10,000,000, and even more preferred are polymers with molecular weights of about 5,000,000 to about 8,000,000. Preferred poly(ethylene oxide)s are those with a weight-average molecular weight of about 1×105 to about 1×107, and preferably within the range of about 9×105 to about 8×106. Poly(ethylene oxide)s are often characterized by their viscosity in solution. A preferred viscosity is about 50 to about 2,000,000 centipoise for a 2% aqueous solution at 20° C. Two specific example of poly(ethylene oxide)s are POLYOX® NF, grade WSR Coagulant, molecular weight 5 million, and grade WSR 303, molecular weight 7 million, both available from Dow.

Polysaccharide gums, both natural and modified (semi-synthetic) can be used. Examples are dextran, xanthan gum, gellan gum, welan gum and rhamsan gum.

Crosslinked polyacrylic acids of greatest utility are those whose properties are the same as those described above for alkyl-substituted cellulose and polyalkylene oxide polymers. Preferred crosslinked polyacrylic acids are those with a viscosity of about 4,000 to about 40,000 centipoise for a 1% aqueous solution at 25° C. Three specific examples are CARBOPOL® NF grades 971P, 974P and 934P (BFGoodrich Co., Specialty Polymers and Chemicals Div., Cleveland, Ohio, USA). Further examples are polymers known as WATER LOCK®, which are starch/acrylates/acrylamide copolymers available from Grain Processing Corporation, Muscatine, Iowa, USA.

The hydrophilicity and water swellability of these polymers cause the active agent-containing matrices to swell in size in the gastric cavity due to ingress of water in order to achieve a size that will be retained in the stomach when introduced during the fed mode. These qualities also cause the matrices to become slippery, which provides resistance to peristalsis and further promotes their retention in the stomach. The release rate of an active agent from the matrix is primarily dependent upon the rate of water imbibition and the rate at which the active agent dissolves and diffuses from the swollen polymer, which in turn is related to the solubility and dissolution rate of the active agent, the active agent particle size and the active agent concentration in the matrix. Also, because these polymers dissolve very slowly in gastric fluid, the matrix maintains its physical integrity over at least a substantial period of time, in many cases at least 90%, and preferably over 100% of the dosing period. The particles will then slowly dissolve or decompose. Complete dissolution or decomposition may not occur until 24 hours or more after the intended dosing period ceases, although in most cases, complete dissolution or decomposition will occur within 10 to 24 hours after the dosing period.

The dosage forms may include additives that impart a small degree of hydrophobic character, to further retard the release rate of the paroxetine into the gastric fluid. One example of such a release rate retardant is glyceryl monostearate. Other examples are fatty acids and salts of fatty acids, one example of which is sodium myristate. The quantities of these additives when present can vary; and in most cases, the weight ratio of additive to active agent will be about 1:20 to about 1:1, and preferably about 1:8 to about 1:2.

The amount of polymer relative to the paroxetine can vary, depending on the paroxetine release rate desired and on the polymer, its molecular weight, and excipients that may be present in the formulation. The amount of polymer should be sufficient however to retain at least about 40 wt % of the paroxetine within the matrix one hour after ingestion (or immersion in the gastric fluid). Preferably, the amount of polymer is such that at least about 50 wt % of the paroxetine remains in the matrix one hour after ingestion. More preferably, at least about 60 wt %, and most preferably at least about 80 wt %, of the paroxetine remains in the matrix one hour after ingestion. In all cases, however, the paroxetine will be substantially all released from the matrix within about ten hours, and preferably within about eight hours, after ingestion or immersion in simulated gastric fluid, and the polymeric matrix will remain substantially intact until all of the active agent is released by substantially all, it is meant that greater than 95 wt %, preferably greater than 99 wt % of the paroxetine is released. The term “substantially intact” is used herein to denote a polymeric matrix in which the polymer portion substantially retains its size and shape without deterioration due to becoming solubilized in the gastric fluid or due to breakage into fragments or small particles.

The water-swellable polymers can be used individually or in combination. Certain combinations will often provide a more controlled-release of the paroxetine than their components when used individually. An exemplary combination is cellulose-based polymers combined with gums, such as hydroxyethyl cellulose or hydroxypropyl cellulose combined with xanthan gum. Another example is poly(ethylene oxide) combined with xanthan gum.

The benefits of this dosage form will be achieved over a wide range of paroxetine loadings, with the weight ratio of paroxetine to polymer of 0.01:99.99 to about 80:20. Preferred loadings (expressed in terms of the weight percent of active agent relative to total of active agent and polymer) are about 15 wt % to about 80 wt %, or about 30 wt % to about 80 wt %, or about 30 wt % to about 70 wt %. For certain applications, however, the benefits will be obtained with active agent loadings of about 0.01 wt % to about 80 wt %, or about 15 wt % to about 80 wt %.

The dosage forms may find their greatest utility when administered to a subject who is in the digestive state (also referred to as the postprandial or “fed” mode). The postprandial mode is distinguishable from the interdigestive (or fasting) mode by their distinct patterns of gastroduodenal motor activity, which determine the gastric retention or gastric transit time of the stomach contents.

In the interdigestive mode, the fasted stomach exhibits a cyclic activity called the interdigestive migrating motor complex (IMMC). The cyclic activity occurs in four phases:

Phase I is the most quiescent, lasts 45 to 60 minutes, and develops few or no contractions.

Phase II is marked by the incidence of irregular intermittent sweeping contractions that gradually increase in magnitude.

Phase III, which lasts 5 to 15 minutes, is marked by the appearance of intense bursts of peristaltic waves involving both the stomach and the small bowel.

Phase IV is a transition period of decreasing activity which lasts until the next cycle begins.

The total cycle time is approximately 90 minutes, and thus, powerful peristaltic waves sweep out the contents of the stomach every 90 minutes during the interdigestive mode. The IMMC may function as an intestinal housekeeper, sweeping swallowed saliva, gastric secretions, and debris to the small intestine and colon, preparing the upper tract for the next meal while preventing bacterial overgrowth. Pancreatic exocrine secretion of pancreatic peptide and motilin also cycle in synchrony with these motor patterns.

Dissolution Profiles FOR Paroxetine Dosage Forms

The paroxetine dosage forms and dosage forms comprising paroxetine and one or more other active agents may be formulated so that particular dissolution profiles are achieved.

A dosage form that may exhibit a dissolution profile that is substantially identical to that of PAXIL® CR™ in the same dissolution media. Alternatively, the controlled release dosage form may release substantially less than all of the paroxetine or pharmaceutically acceptable salt thereof at five hours after administration to a human. By substantially less than all, it is meant that less than about 85 wt %, preferably less than about 90 wt % of the paroxetine or pharmaceutically acceptable salt thereof is released.

Pharmacokinetic Properties of Paroxetine Dosage Forms

The invention provides the paroxetine dosage forms and dosage forms comprising paroxetine and one or more other active agents (combinations) formulated so that particular plasma levels, Cmax, Tmax, and AUC values are achieved.

A dosage form that may exhibit a Cmax value and AUC from time of administration to 24 hours after administration that are from 80% to 120% of the Cmax value and AUC from time of administration to 24 hours after administration exhibited by PAXIL® CR™ under the same conditions. The mean Cmax for PaxilE CR™ is, for example, 2.0 ng/mL for a 12.5 mg dose, 5.5 ng/mL for a 25 mg dose, 9.0 ng/mL for a 37.5 mg dose, and 12.5 ng/mL for a 50 mg dose. The mean AUC₀₋₂₈ for Paxil® CR™ is, for example, 121 ng-hr/mL for a 12.5 mg dose, 261 ng-hr/mL for a 25 mg dose, 338 ng·hr/mL for a 37.5 mg dose, and 540 ng-hr/mL for a 50 mg dose. The Tmax is about 6 to about 10 hours post-dose.

In one embodiment, the dosage form has less variability in blood levels than of PAXIL® CR™.

Manufacture of Dosage Forms

Amorphous Technology

Amorphous solids consist of disordered arrangements of molecules and do not possess a distinguishable crystal lattice. The active agent may be prepared in such a way that substantially all of the paroxetine is present in amorphous form.

A process for preparing solid, amorphous paroxetine comprises mixing paroxetine free base or a pharmaceutically acceptable salt thereof with a solvent such as water and a pharmaceutically acceptable polymeric carrier; and drying to form a composition comprising amorphous paroxetine and polymeric carrier.

In another aspect, a pharmaceutical composition comprises paroxetine hydrochloride in amorphous, solid form, and polymeric carrier, prepared by the aforementioned process.

Suitable pharmaceutically acceptable polymeric carriers include, for example, hydroxypropyl cellulose, methyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polypropylene, dextrans, dextrins, hydroxypropyl-beta-cyclodextrin, chitosan, co(lactic/glycolid) copolymers, poly(orthoester), poly(anhydrate), polyvinyl chloride, polyvinyl acetate, ethylene vinyl acetate, lectins, carbopols, silicon elastomers, polyacrylic polymers, maltodextrins, polyvinylpyrrolidone (PVP), polyethylene glycol (PEG), and alpha-, beta-, and gamma-cyclodextrins, and combinations comprising one or more of the foregoing carriers.

Suitable polymeric carriers are one or more of polyvinylpyrrolidone, hydroxypropylmethyl cellulose, hydroxypropyl cellulose, methyl cellulose, block co-polymers of ethylene oxide and propylene oxide, and polyethylene glycol, wherein a more preferred polymeric carrier is polyvinylpyrrolidone (PVP) having an average molecular weight of about 2,500 to about 3,000,000, more preferably polyvinylpyrrolidone having an average molecular weight of about 10,000 to about 450,000.

The polymeric carrier is preferably miscible with both the paroxetine free base and the salt, capable of keeping the salt in a homogeneous noncrystalline solid state dispersion after the solvent has been removed by evaporation and chemically inert with respect to the free base of the active ingredient, the salt of the free base, and the acid solution.

The paroxetine may be added in either free base or salt form. When the paroxetine is added in free base form, the process comprises adding an acid corresponding to a pharmaceutically acceptable salt of the paroxetine to the mixture or solution of the free base. The free base is then converted to a salt in situ, for example by addition of an inorganic or an organic acid. The acid may be added either as a gas, a liquid or as a solid dissolved into the solvent. A preferred acid is hydrogen chloride and the molar quantity of acid added to the solution of paroxetine free base and carrier may either be in stoichiometric proportion to the paroxetine free base or be in excess of the molar quantity of the paroxetine free base, especially when added as a gas.

In one embodiment, hydrogen chloride added is about 1.0 to about 1.8 times the molar quantity of paroxetine free base. Preferred molar ratios of paroxetine to HCl are about 1:1 to 1:1.8, or about 1:1.1. Although hydrogen chloride is readily added as a gas, the preferred method to add the hydrogen chloride in the form of hydrogen chloride dissolved into the solvent. It is understood that upon addition of the acid, the formed free base salt remains dissolved in solution with the polymeric carrier.

The paroxetine, polymeric carrier, and solvent may be combined in a manner so as to form a solution of paroxetine hydrochloride and the polymeric carrier.

In forming a solution of polymeric carrier and solvent, heating of the solution is not necessary at lower concentrations, but is preferred at higher concentrations, provided that the temperature does not result in decomposition or degradation of any materials. It is preferred to add the active agent free base or salt after dissolving the polymeric carrier in solvent, suitably at about 25° C. to about 100° C., or at about 45° C. to about 80° C. When the paroxetine is added as a free base, it is preferred to form a salt at a temperature at which the final solution is clear. A temperature of at least about 60° C. may result in a clear solution of the paroxetine hydrochloride being formed, although for other concentrations clear solutions are formed at other temperatures. It is preferred to only add enough heat to form a clear solution.

The ratio of paroxetine to the polymeric carrier can be varied over a wide range and depends on the concentration of paroxetine required in the pharmaceutical dosage form ultimately administered. The ratio by weight of polymeric carrier to paroxetine hydrochloride is about 20:1 to about 0.5:1; or about 4:1 to about 1:1; or about 3:1 to about 1.5:1; or about 2:1.

Upon formation of the clear solution, the process proceeds by removing the solvent to form a solid state dispersion of the free base salt in the polymeric carrier. A method of removal of the solvent which renders a homogeneous solid state dispersion is intended, although preferred are methods of evaporation under vacuum or spray drying. Methods of evaporation under vacuum include rotary evaporation, static vacuum drying, and combination thereof. It is understood that one skilled in the art of pharmaceutical formulations can determine a reasonable temperature at which solvent can be removed, provided the temperature is not so high as to cause degradation or decomposition of the materials; however, it is preferred that evaporation occurs at about 25° C. to about 100° C. Evaporation of solvent should render a solid state dispersion which is homogeneous and substantially free of solvent. By substantially free it is meant that the solid state dispersion contains less than about 20 wt % by weight of residual solvent, or less than about 10 wt %, or less than about 5 wt %, or less than about 1 wt %.

The ratio of paroxetine free base to the polymeric carrier can be varied over a wide range and depends on the concentration of active agent required in the pharmaceutical dosage form ultimately administered. However, the preferred range of active agent in the solid dispersion is about 10 wt % to about 50 wt % of the total solid dispersion weight, more preferable is about 20 wt % to about 50 wt %, even more preferable is about 25 wt % to about 40 wt %, most preferable is about 33 wt % of the total dispersion weight.

Suitable pharmaceutically acceptable excipients can be added in the process. Examples of pharmaceutically acceptable excipients include diluents, binders, disintegrants, coloring agents, flavoring agents, lubricants and/or preservatives. The pharmaceutical composition may be formulated by conventional methods of admixture such as blending, filling, granulation and compressing. These agents may be utilized in conventional manner.

Optional Additional Additives

Excipients

Excipients are components added to paroxetine pharmaceutical formulation other than the active agent. Excipients may be added to facilitate manufacture, enhance stability, control release, enhance product characteristics, enhance bioavailability, enhance patient acceptability, etc. Pharmaceutical excipients include binders, disintegrants, lubricants, glidants, compression aids, colors, sweeteners, preservatives, suspending agents, dispersing agents, film formers, flavors, printing inks, etc. Binders hold the ingredients in the dosage form together. Exemplary binders include, for example, polyvinyl pyrrolidone, hydroxypropyl cellulose, hydroxypropyl methylcellulose, methylcellulose and hydroxyethyl cellulose, sugars, and combinations comprising one or more of the foregoing binders. Disintegrants expand when wet causing a tablet to break apart. Exemplary disintegrants include water swellable substances, for example, low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose (sodium croscarmellose), e.g. Ac-di-sol®, Primellose®; sodium starch glycolate, e.g. Primojel®; sodium carboxymethylcellulose, e.g. Nymcel ZSB 10® sodium carboxymethyl starch, e.g. Explotab®; ion-exchange resins, e.g. Dowex® or Amberlite®; microcrystalline cellulose, e.g. Avicel®; starches and pregelatinized starch, e.g. Starch 1500®, Sepistab ST200®; formalin-casein, e.g. Plas-Vita®, and combinations comprising one or more of the foregoing water swellable substances. Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. Glidants include, for example, silicon dioxide.

Fillers

Certain dosage forms described herein contain a filler, such as a water insoluble filler, water soluble filler, and combinations thereof. The filler may be a water insoluble filler, such as silicon dioxide, titanium dioxide, talc, alumina, starch, kaolin, polacrilin potassium, powdered cellulose, microcrystalline cellulose, and combinations comprising one or more of the foregoing fillers. Exemplary water-soluble fillers include water soluble sugars and sugar alcohols, preferably lactose (e.g., lactose monohydrate NF), glucose, fructose, sucrose, mannose, dextrose, galactose, the corresponding sugar alcohols and other sugar alcohols, such as mannitol, sorbitol, xylitol, and combinations comprising one or more of the foregoing filler

Preparation of the Active Agent

Preparation of Subunits

The paroxetine and any optional additives may be prepared in many different ways, for example as subunits. Pellets comprising paroxetine can be prepared, for example, by a melt pelletization technique. In this technique, the paroxetine in finely divided form is combined with a binder and other optional inert ingredients, and thereafter the mixture is pelletized, e.g., by mechanically working the mixture in a high shear mixer to form the pellets (e.g., pellets, granules, spheres, beads, etc., collectively referred to herein as “pellets”). Thereafter, the pellets can be sieved in order to obtain pellets of the requisite size. The binder material may also be in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like, and combinations comprising one or more of the foregoing binders.

Oral dosage forms may be prepared to include an effective amount of melt-extruded subunits containing the paroxetine and/or other optional active agents in the form of multiparticles within a capsule. For example, a plurality of the melt-extruded multiparticulates can be placed in a gelatin capsule in an amount sufficient to provide an effective release dose when ingested and contacting by gastric fluid.

Subunits, e.g., in the form of multiparticulates, can be compressed into an oral tablet using conventional tableting equipment using standard techniques. The tablet formulation may include excipients such as, for example, an inert diluent such as lactose, granulating and disintegrating agents such as cornstarch, biding agents such as starch, and lubricating agents such as magnesium stearate.

Alternatively, the subunits containing the paroxetine and optionally containing additional active agents are added during the extrusion process and the extrudate can be shaped into tablets by methods know in the art. The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and include a plurality of subunits, preferably within a range of similar size and/or shape. The melt-extruded multiparticulates can be about 0.1 to about 12 mm in length and have a diameter of about 0.1 to about 5 mm. In addition, the melt-extruded multiparticulates can be any geometrical shape within this size range. Alternatively, the extrudate can simply be cut into desired lengths and divided into unit doses of the therapeutically active agent without the need of a spheronization step.

The melt-extruded dosage forms can further include combinations of melt-extruded multiparticulates containing one or more of the therapeutically active agents before being encapsulated. Furthermore, the dosage forms can also include an amount of the paroxetine formulated for immediate-release for prompt therapeutic effect. The paroxetine formulated for immediate-release can be incorporated or coated on the surface of the subunits after preparation of the dosage forms (e.g., controlled-release coating or matrix-based). The dosage forms can also contain a combination of controlled-release beads and matrix multiparticulates to achieve a desired effect.

A melt-extruded material may be prepared without the inclusion of subunits containing the paroxetine, which are added thereafter to the extrudate. Such formulations have the subunits and other active agents blended together with the extruded matrix material. The mixture is then tableted in order to provide release of the paroxetine or other active agents. Such formulations can be particularly advantageous, for example, when an active agent included in the formulation is sensitive to temperatures needed for softening the hydrophobic material and/or the retardant material.

The oral dosage form containing the paroxetine may be in the form of micro-tablets enclosed inside a capsule, e.g. a gelatin capsule. For this, a gelatin capsule as is employed in pharmaceutical formulations can be used, such as the hard gelatin capsule known as CAPSUGEL, available from Pfizer.

Particles

Many of the oral dosage forms described herein contain the paroxetine and optionally additional active agents in the form of particles. Such particles may be compressed into a tablet, present in a core element of a coated dosage form, such as a taste masked dosage form, a press coated dosage form, or an enteric coated dosage form, or may be contained in a capsule, osmotic pump dosage form, or other dosage form.

For particles, such as powder particles, present in the core element of a coated dosage form, the core element may have a particle size distribution with a median of about 100 μm. The particles in the distribution may vary from about 1 μm to about 250 μm, or about 25 μm to about 250 μm, or about 35 μm to about 125 μm. If the median of the distribution is close to either extreme of the distribution, the taste masking or sustained-release characteristics may be affected. In a particle size range of about 25 μm to about 250 μm, for example, no more than about 25% of particles can be less than about 25 μm, and no more than about 25% can be over about 250 μm.

Another parameter to consider is particle shape. Particle shape can influence the coverage and stability of the coat. Both the crystallinity of the active agent and the aspect ratio of the particles are related to particle shape. It is preferred that the paroxetine in the coated dosage forms has a crystalline morphology, however, sharp angles on a crystal can cause weaknesses in the coat. These sharp corners may lead to stress points on the coat and cause weaknesses in the structure possibly leading to premature release of the active agent from the dosage form. Furthermore, areas of thin coating are susceptible to breaking and cracking and hence ineffective for sustained-release and taste masking.

Regarding the aspect ratio, a low aspect ratio may be employed. The aspect ratio is a measure of the length to breadth. For example, a low aspect ratio of about 1 would be a box or sphere. Crystals with a high aspect ratio are more pointed with needle-like crystals. Crystals with a high aspect ratio may result in a relatively thin coat at the crystal needle tips leading to a more rapid release rate of the active agent than is preferred. A low aspect ratio spherical shape of the particle is advantageous for both solubility of the coat and high payload of the active agent. Therefore, it is most preferable that the aspect ratio is less than about 3, or about 1 to about 2, or approximately 1 providing a substantially rounded shape.

Inconsistencies in size and shape can lead to inconsistent coating. Where the particles containing the paroxetine are of different size and shape, polymeric coating materials such as ethyl cellulose may deposit differently on each particle. It is therefore preferable for coated dosage forms that substantially all particles of the dosage form have substantially the same size and shape so that the coating process is better controlled and maintained.

Preparartion of Dosage Forms

Controlled-release dosage forms of paroxetine and its pharmaceutically acceptable salts preferably comprise 12.5 mg, 25 mg or 35 mg of the paroxetine or pharmaceutically acceptable salt.

The term “dosage form” denotes a form of a formulation that contains an amount sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics such as solubility, and with the characteristics of the swellable matrix such as its permeability, and the relative amounts of the drug and polymer. In most cases, the dosage form will be such that effective results will be achieved with administration no more frequently than once every eight hours or more, preferably once every twelve hours or more, and even more preferably once every twenty-four hours or more.

The dosage form can be prepared by various conventional mixing, comminution and fabrication techniques readily apparent to those skilled in the chemistry of drug formulations. Examples of such techniques are as follows:
(1) Direct compression, using appropriate punches and dies; the punches and dies are fitted to a suitable rotary tableting press;
(2) Injection or compression molding using suitable molds fitted to a compression unit
(3) Granulation followed by compression; and
(4) Extrusion in the form of a paste, into a mold or to an extrudate to be cut into lengths.

When particles are made by direct compression, the addition of lubricants may be helpful and sometimes important to promote powder flow and to prevent capping of the particle (breaking off of a portion of the particle) when the pressure is relieved. Useful lubricants are magnesium stearate (in a concentration of 0.25 wt % to 3 wt % by weight, preferably less than 1 wt %, in the powder mix), and hydrogenated vegetable oil (preferably hydrogenated and refined triglycerides of stearic and palmitic acids at about 1 wt % to 5 wt %, most preferably about 2 wt %. Additional excipients may be added to enhance powder flowability and reduce adherence.

Pellets in Capsules

Oral dosage forms may be prepared to include an effective amount of melt-extruded subunits in the form of multiparticles within a capsule. For example, a plurality of the melt-extruded multiparticulates can be placed in a gelatin capsule in an amount sufficient to provide an effective release dose when ingested and contacted by gastric fluid.

Pellets in Tablets

The subunits, e.g., in the form of multiparticulates, can be compressed into an oral tablet using conventional tableting equipment using standard techniques. Techniques and compositions for making tablets (compressed and molded), capsules (hard and soft gelatin) are also described in Remington's Pharmaceutical Sciences, (Aurther Osol., editor), 1553-1593 (1980).

Tablets in Capsules

The composition may be in the form of micro-tablets enclosed inside a capsule, e.g., a gelatin capsule. For this, a gelatin capsule employed in the pharmaceutical formulation field can be used, such as the hard gelatin capsule known as CAPSUGEL, available from Pfizer.

Manufacturing of Tablets

Manufacturing problems may be associated with high dosage forms of an active agent, such as suitable compression and moisture, especially in the manufacture of tablets. For example, many active agents require carefully controlled amounts of water to be present during tablet compression to control capping. Capping denotes the detachment of layers of compressed mass during the pressing or shortly thereafter. Capping can be caused by any number of problems, including inadequate binding agent action, inadequate or excessive moisture content of the granulate, unsuitable crystal forms, strongly aerophilic substances, excessive porosity, excessive proportion of powder, excessive interparticulate binding between the granulate particles and unsuitable granulate forms. Machine factors may also lead to capping, including excessive pressing force, badly applied or worn tools, excessive pressing rages and poor deaeration of the matrix (fixed pressure). However, in the case of high dose active agents, the usual measures are often inadequate to suitably control the capping of the tableting mass.

Coatings

The formulations described herein may be coated with a functional or non-functional coating. The coating may comprise 0 wt % to about 40 wt % of the composition. The coating material may include a polymer, preferably a film-forming polymer, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly (ethyl methacrylate), poly (butyl methacrylate), poly (isobutyl methacrylate), poly (hexyl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene)high density, (poly propylene), poly (ethylene glycol poly (ethylene oxide), poly (ethylene terephthalate), poly(vinyl alcohol), poly(vinyl isobutyl ether), poly(viny acetate), poly (vinyl chloride), polyvinyl pyrrolidone, and combinations comprising one or more of the foregoing polymers.

In applications such as taste-masking, the polymer can be a water-insoluble polymer. Water insoluble polymers include ethyl cellulose or dispersions of ethyl cellulose, acrylic and/or methacrylic ester polymers, cellulose acetates, butyrates or propionates or copolymers of acrylates or methacrylates having a low quaternary ammonium content, and the like, and combinations comprising one or more of the foregoing polymers.

In controlled-release applications, for example, the coating can be a hydrophobic polymer that modifies the release properties of the paroxetine from the formulation. Suitable hydrophobic or water insoluble polymers for controlled-release include, for example, methacrylic acid esters, ethyl cellulose, cellulose acetate, polyvinyl alcohol-maleic anhydride copolymers, β-pinene polymers, glyceryl esters of wood resins, and combinations comprising one or more of the foregoing polymers.

The inclusion of an effective amount of a plasticizer in the coating composition may improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 wt % to about 50 wt % of the polymer. Concentrations of the plasticizer, however, can be determined by routine experimentation.

Examples of plasticizers for ethyl cellulose and other celluloses include plasticizers such as dibutyl sebacate, diethyl phthalate, triethyl citrate, tributyl citrate, triacetin, and combinations comprising one or more of the foregoing plasticizers, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, and combinations comprising one or more of the foregoing plasticizers, although it is possible that other plasticizers (such as acetylated monoglycerides, phthalate esters, castor oil, etc.) can be used.

An example of a functional coating comprises a coating agent comprising a poorly-water-permeable component (a) such as, an alkyl cellulose, for example an ethylcellulose, such as AQUACOAT (a 30% dispersion available from FMC, Philadelphia, Pa.) or SURELEASE (a 25% dispersion available from Colorcon, West Point, Pa.) and a water-soluble component (b), e.g., an agent that can form channels through the poorly-water-permeable component upon the hydration or dissolution of the soluble component. Preferably, the water-soluble component is a low molecular weight, polymeric material, e.g., a hydroxyalkylcellulose, hydroxyalkyl(alkylcellulose), and carboxymethylcellulose, or salts thereof. Particular examples of these water soluble polymeric materials include hydroxyethylcellulose, hydroxypropylcellulose, hydroxyethylmethylcellulose, hydroxypropyl methylcellulose, carboxymethylcellulose, sodium carboxymethylcellulose, and combinations comprising one or more of the foregoing materials. The water-soluble component can comprise hydroxypropyl methylcellulose, such as METHOCEL (Dow). The water-soluble component is preferably of relatively low molecular weight, preferably less than or equal to about 25,000 molecular weight, or preferably less than or equal to about 21,000 molecular weight.

In the functional coating, the total of the water soluble portion (b) and poorly-water permeable portion (a) are present in weight ratios (b):(a) of about 1:4 to about 2:1, preferably about 1:2 to about 1:1, and more preferably in a ratio of about 2:3. While the ratios disclosed herein are preferred for duplicating target release rates of presently marketed dosage forms, other ratios can be used to modify the speed with which the coating permits release of the active agent. The functional coating may comprise about 1 wt % to about 40 wt %, preferably about 3 wt % to about 30 wt %, more preferably about 5 wt % to about 25 wt %, and yet more preferably about 6 wt % to about 10 wt % of the total formulation.

In certain embodiments, particularly where the coating provides taste masking, it is preferred that the coating is substantially continuous coat and substantially hole-free. By substantially continuous coating is meant a coating which retains a smooth and continuous appearance when magnified 1000 times under a scanning electron microscope and wherein no holes or breakage of the coating are evident.

Suitable methods can be used to apply the coating to the paroxetine. Processes such as simple or complex coacervation, interfacial polymerization, liquid drying, thermal and ionic gelation, spray drying, spray chilling, fluidized bed coating, pan coating, electrostatic deposition, may be used. A substantially continuous nature of the coating may be achieved, for example, by spray drying from a suspension or dispersion of the paroxetine in a solution of the coating composition including a polymer in a solvent in a drying gas having a low dew point.

When a solvent is used to apply the coating, the solvent is preferably an organic solvent that constitutes a good solvent for the coating material, but is substantially a non-solvent or poor solvent for of the paroxetine. While the paroxetine may partially dissolve in the solvent, it is preferred that the paroxetine will precipitate out of the solvent during the spray drying process more rapidly than the coating material. The solvent may be selected from alcohols such as methanol, ethanol, halogenated hydrocarbons such as dichloromethane (methylene chloride), hydrocarbons such as cyclohexane, and combinations comprising one or more of the foregoing solvents. Dichloromethane (methylene chloride) has been found to be particularly suitable.

The concentration of polymer in the solvent will normally be less than about 75 wt %, and typically about 10 wt % to about 30 wt %. After coating, the coated dosage forms may be allowed to cure for at least about 1 to about 2 hours at a temperature of about 50° C. to about 60° C., more preferably of about 55° C.

The coatings may be about 0.005 micrometers to about 25 micrometers thick, preferably about 0.05 micrometers to about 5 micrometers.

### EXAMPLES

The following examples further illustrate the invention but, of course, should not be construed as in any way limiting its scope.

### Example 1

### Production of Paroxetine Tablet Cores

Paroxetine hydrochloride tablet core were formed according to Table 1

TABLE 1

| | 12.5 mg tablet | | 25 mg tablet | | 37.5 mg tablet | |
|---|---|---|---|---|---|---|
| | mg per | g per | mg per | g per | mg per | G per |
| Component | tablet | batch | tablet | batch | tablet | batch |
| Paroxetine | 14.225 | 3,414 | 28.45 | 6,259 | 42.675 | 8,535 |
| Hydrochloride | | | | | | |
| Lactose | 169.775 | 40,746 | 169.55 | 37,301 | 169.375 | 33,865 |
| monohydrate | | | | | | |
| NF (impalp- | | | | | | |
| able) | | | | | | |
| Hydroxyethyl | 14 | 3,360 | 15 | 3,300 | 16 | 3,200 |
| cellulose | | | | | | |
| Magnesium | 2 | 480 | 2 | 440 | 2 | 400 |
| stearate | | | | | | |
| TOTAL | 200 | 48,000 | 215 | 47,300 | 230 | 46,000 |

The paroxetine hydrochloride was wet granulated with the lactose monohydrate and hydroxyethyl cellulose or hypromellose with 6,000 (12.5 mg tablet) or 12,000 (25, 37.5 mg tablets) grams of water. The wet granulate was passed through a 3 mesh screen and then dried in a fluid bed drier. The granulate was then milled and passed through a 40 mesh screen. The dried and screened granulate was mixed with magnesium stearate to form a final blend. The final blend was suitable for compression into tablets containing 12.5 mg paroxetine and having a unit weight of 200 mg and a core tablet hardness of 8.0 kp to 16.0 kp.

### Example 2

### Enteric Coating of Paroxetine Tablet Cores

Tablet cores formed according to example 1 were enteric coated using the components listed in Table 2. Coating of the 37.5 mg tablet is used as an example.

TABLE 2

| Component | Amount per tablet | Amount per batch |
|---|---|---|
| 37.5 mg paroxetine | 230 | |
| hydrochloride tablet core | | |
| Eudragit L30 D-55 (30% | 14.35 | 12,915 |
| solids)* | | |
| Triethyl citrate | 1.45 | 391 |
| Talc, USP | 7.2 | 1,944 |
| Total | 253 | 51,750 |

*Eudragit L30 D-55 contains methacrylic acid copolymer (Type C), sodium lauryl sulfate, polysorbate 80 and purified water.

The Eudragit, triethyl citrate, talc and 36,500 grams of water were mixed to form the enteric coating solution. The enteric coating solution was then sprayed onto the paroxetine hydrochloride tablet cores.

### Example 3

### Film Coating of Enteric Coated Cores

The enteric coated cores were then coated with one or more colored and/or clear film coats comprising Opadry and water. Opadry contains a combination of hypromellose and polyethylene glycol.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A controlled-release dosage form comprising
a pharmaceutically effective amount of paroxetine or a pharmaceutically acceptable salt thereof, and
a hydrophilic release-retarding material, wherein the hydrophilic release-retarding material is not hydroxypropyl methylcellulose.

2. The controlled-release dosage form of claim 1, wherein the hydrophilic release-retarding material is an acrylic polymer, an alkylcellulose other than hydroxypropyl methylcellulose, shellac, zein, hydrogenated vegetable oil, hydrogenated castor oil, or a combination comprising one or more of the foregoing materials.

3. The controlled release dosage form of claim 2, wherein the release retarding material is hydroxyethyl cellulose.

4. The controlled release dosage form of claim 3, further comprising lactose monohydrate NF.

5. The controlled-release dosage form of claim 1, wherein the pharmaceutically acceptable salt of paroxetine is paroxetine hydrochloride.

6. The controlled-release dosage form of claim 1, wherein the dosage form further comprises an enteric coating.

7. The controlled release dosage form of claim 6, wherein the enteric coating comprises a methacrylic acid/methacrylate polymer and triethyl citrate.

8. The controlled-release dosage form of claim 1, wherein the dosage form releases substantially less than all of the paroxetine or pharmaceutically acceptable salt thereof at five hours after administration to a human.

9. The controlled-release dosage form of claim 1, wherein the dosage form exhibits less variability in blood levels than a comparable dose of Paxil® CR™.

10. The controlled-release dosage form of claim 1, wherein bioequivalence to Paxil® CR™ is provided according to FDA guidelines or criteria.

11. The controlled-release dosage form of claim 1, which provides an AUC between 0 and 24 hours after administration that is more than 80 percent and less than 120 percent of the AUC provided by 2 times the equivalent weight of Paxil® CR™ Paxil CR between 0 and 12 hours after administration.

12. A method of forming a controlled-release dosage form comprising wet granulating paroxetine or a pharmaceutically acceptable salt thereof and a release-retarding material.

13. The method of claim 12, wherein the release-retarding material is not hydroxypropyl methylcellulose.

14. The method of claim 13, wherein the hydrophilic release-retarding material is an acrylic polymer, an alkylcellulose other than hydroxypropyl methylcellulose, shellac, zein, hydrogenated vegetable oil, hydrogenated castor oil, or a combination comprising one or more of the foregoing materials.

15. The method of claim 14, wherein the release retarding material is hydroxyethyl cellulose.

16. The method of claim 15, wherein the dosage form further comprises lactose monohydrate NF.
